(19) United States
(12) Patent Application Publication
(10) Pub. No.: US 2022/0304940 A1
(43) Pub. Date: Sep. 29, 2022

(54) POLYMERIC SOFT FILMS EMBEDDED WITH NANODOMAINS AND/OR A BIOACTIVE AND METHODS OF PRODUCING SAME

(71) Applicant: LYOTROPIC DELIVERY SYSTEMS LTD., Jerusalem (IL)

(72) Inventors: Nissim GARTI, Ramat Hasharon (IL); Sharon GARTI-LEVI, Modiin (IL); Gal AIMEE NOKREAN, Jerusalem (IL); Rotem EDRI, Eilat (IL)

(73) Assignee: LYOTROPIC DELIVERY SYSTEMS LTD., Jerusalem (IL)

(21) Appl. No.: 17/596,849

(22) PCT Filed: May 25, 2020
(86) PCT No.: PCT/IL2020/050571

§ 371 Date: Dec. 20, 2021

Related U.S. Application Data
(60) Provisional application No. 62/863,994, filed on Jun. 20, 2019.

(51) Int. Cl. A61K 9/70; A61K 9/00

(52) U.S. Cl. CPC A61K 9/7007; A61K 9/0014

(57) ABSTRACT

Provided is a drug delivery system including a soft hydrophilic film layer and a plurality of nanodomains embedded within the film layer, wherein the film layer, having embedded therein the nanodomains, is transparent or translucent, has a thickness of less than 1000 microns, wherein the nanodomains have an average size of less than about of 100 nm, and wherein the nanodomains maintain their structural integrity within the film when stored at room temperature for 1 month or more.

### TECHNOLOGICAL FIELD

The present disclosure is directed to formation of thin, porous and soft biopolymeric films embedded with bioactive-loaded nanodomains, serving as controlled delivery systems suitable for topical and transdermal applications.

### BACKGROUND

Application of nanosized liquid nanodomain vehicles as drug delivery systems are gaining significant interest due to their high solubilization (loading) capacity, making them of particular interest as carriers for drugs, botanicals, cosmetics, cosmeceuticals and/or nutraceuticals (collectively referred to herein as APIs). Liquid nanodomains differ from emulsions, mini or nano emulsions (which are non-stable thermodynamically), liposomes, lyotropic liquid crystals (reverse hexagonal mesophases-Hii, cubic liquid crystals, or related structures such as hexosomes, cubosomes, ethosomes, ribbons, cubic liquid-Q1), and others. The nanodomains have a large surface area due to their nanosizes and are almost mono dispersed in nature, and unlike microemulsions, they are designed to better adhere to physiological membranes, or biological or human tissue surfaces and thereby enable and enhance permeation and the delivery of APIs across membranes into skin layers as well as for systemic delivery.

### SUMMARY OF THE INVENTION

The following embodiments and aspects thereof are described and illustrated in conjunction with compositions and methods which are meant to be exemplary and illustrative, not limiting in scope. In various embodiments, one or more of the above-described problems have been reduced or eliminated, while other embodiments are directed to other advantages or improvements.

According to some embodiments, there is provided a drug delivery system comprising a polymeric film or film layer, having embedded therein a plurality of nanodomains with an average size of less than about 50 nm. Advantageously, the film or film layer, having embedded therein the nanodomains (after casting and drying) is thin and soft and has a thickness of about 200 microns-1000 microns. According to some embodiments, the film, or film layer has a Young's Modulus elasticity of below 1.5 MPa. According to some embodiments, the film or film layer has a tensile strength at breaking point of 1.5 or below MPa. According to some embodiments, the film or film layer is a transparent, or translucent (light transmission of more than 65%), drug delivery system. Advantageously, the film or film layer is, when applied, essentially invisible to the naked eye. This is of particular importance when the treatment area is visible (e.g. facial, vaginal, buccal treatment) and thus significantly improves patient compliance.

According to some embodiments, the nanodomains may be loaded with an active pharmaceutical ingredient (API). The film or film layer, embedded with the nanodomains loaded with API, is configured to controllably release the nanodomains and/or the API, thereby enabling delivery of the API across membranes with minimal drug being retained in the film upon use.

According to some embodiments, the film or film layer may enable release of at least 50%, at least 60%, least 70%, at least 80% or at least 90% of the nanodomains and/or API within less than one week from being applied. Each possibility is a separate embodiment.

According to some embodiments, the film or film layer may enable release of 0.01%-15% of the nanodomains and/or API within 24 h from being applied. According to some embodiments, the film or film layer may enable release of 0.1%-15% of the nanodomains and/or API within 24 h from being applied. According to some embodiments, the film or film layer may enable release of 1%-15% of the nanodomains and/or API within 24 h from being applied.

According to some embodiments, the composition of the nanodomains is specifically tailored to enable their embedding within the polymeric film without compromising the structural integrity of the nanodomains, or the properties of the film.

Advantageously and surprisingly, the nanodomains disclosed herein maintain their structural integrity within the film to such an extent that when stored at room temperature, release of the API from the film during storage is essentially prevented during at least 1-12 months of storage at room temperature. That is, the films of the herein disclosed drug delivery system serve as a reservoir for the specially designed nanodomains (different from the classical micro emulsions, double emulsions, emulsified microemulsions, mini emulsions, lyotropic liquid crystals liposomes, or nano emulsions and others) which, as demonstrated herein below, do not disrupt the nanodomains embedded therein.

Importantly, the nanodomains and/or the API embedded in the nanodomains are, for example, upon contact with a subject's skin or other tissues, released from the film, or film layer, in a slow and controlled manner. This indicates that the nanodomains remain structured within the film, or film layer, and that they are homogeneously dispersed within the film. As demonstrated herein below, following disintegration/dissolution of the film, or discharge (release of API) from the film, the nanodomains remain structured (retain their size and shape) or are spontaneously reformed, restructured or reconstituted (no energy is involved). This also indicates that the nanodomains, even after being casted and the film dried, maintain their integrity within the film, or spontaneously reform once released from the film.

An additional advantage of the herein disclosed film, embedded with nanodomains is applicable for both topical (dermal) and transdermal delivery of APIs. According to some embodiments, depending on the polymer and nanodomains used, the film may be specifically suitable for topical delivery of APIs or for transdermal delivery of APIs. According to some embodiments, the films can be specifically designed to provide a desired release profile (e.g. transdermal/topical and slow/fast release) and may thus be customized, based on API-demands and/or the disorder treated.

As a non-limiting example, the film embedded with the nanodomains may be suitable for transdermal delivery of an API (such as, but not limited to cannabinoids, CBD and THC, Acyclovir, calcitonin) for the treatment of psoriasis. According to some embodiments, the film may be used in the treatment of psoriasis.

As another non-limiting example, the film embedded with the nanodomains may be suitable for topical delivery of an API, such as, but not limited to UV radiation blockers (e.g. astaxanthin), ibuprofen for pain relief, terbinafine and/or ketoconazole for treatment of onychomycosis, minocycline and/or doxycycline for treatment of acne and other inflammation, hyaluronic acid for wound healing and wrinkle treatment.

The herein disclosed film/film layer embedded with bioactive-loaded nanodomains can serve as a novel and advantageous drug delivery system.

According to some embodiments, there is provided a drug delivery system comprising a polymeric film layer and a plurality of nanodomains embedded within the film layer. According to some embodiments, the polymer may include a polymer blend.

According to some embodiments, the nanodomains may be loaded with an API. According to some embodiments, the film layer is configured to release the API and/or the nanodomains upon being adhered to tissue.

According to some embodiments, the film or film layer, having embedded therein the nanodomains, is transparent or translucent. According to some embodiments, the film is transparent. According to some embodiments, the film or film layer has a thickness of less than about 1000 microns, less than about 800 microns, or less than about 500 microns. According to some embodiments, the film or film layer has a Young's Modulus elasticity of about 5 MPa or below, 3 MPa or below or 1.5 MPa or below. Each possibility is a separate embodiment. According to some embodiments, the film or film layer has a Young's Modulus elasticity in a range of about 0.1 KPa to about 5 MPa. According to some embodiments, the film or film layer has a Young's Modulus elasticity in a range of about 0.1 KPa-3 MPa. According to some embodiments, the film or film layer has a Young's Modulus elasticity in a range of about 0.1 MPa-1.5 MPa. According to some embodiments, the film has a Young's Modulus elasticity in a range of about 0.1 MPa-1.0 MPa. According to some embodiments, the film or film layer has a tensile strength at breaking point of about 2 MPa or below. According to some embodiments, the film or film layer has a tensile strength at breaking point in a range of about 1.5 MPa or below. According to some embodiments, the film has a tensile strength at breaking point in a range of about 0.2 KPa-1.5 MPa. According to some embodiments, the film has a tensile strength at breaking point in a range of about 0.2 KPa-1 MPa. The nanodomains have an average size of less than about 100 nm and maintain their structural integrity within the film when stored at room temperature for 1 month or more, such that release of the API from the film during storage is essentially prevented.

According to some embodiments, the nanodomains comprise 20-60% of the total dry weight of the drug delivery film system. According to some embodiments, the film polymer comprises 10-60 wt % of the total dry weight of the drug delivery system.

According to some embodiments, the film comprises a plasticizer. According to some embodiments, the plasticizer is selected from glycerol, sorbitol or other polyols or mono/di and poly-carboxylic acids. According to some embodiments, the plasticizer comprises glycerol and/or sorbitol. According to some embodiments, the plasticizer comprises isosorbide, sorbitan esters, citrates, phosphate esters, dibenzoates, benzoates, azelates, sebacates or any combination thereof. Each possibility is a separate embodiment.

According to some embodiments, the plasticizer may be a substantially non-volatile organic substance (mainly liquids). According to some embodiments, the plasticizer, when incorporated into a plastic or elastomer, improves the polymer's flexibility, extensibility and, processability. According to some embodiments, the plasticizer may increase the flow and thermo-plasticity of the polymer(s) by decreasing the viscosity of the polymer melt, the glass transition temperature (Tg), the melting temperature (Tm) and the elastic modulus of the finished product without altering the fundamental chemical character of the plasticized material.

According to some embodiments, the polymeric film layer further comprises an antibacterial agent. According to some embodiments, the antibacterial agent is dispersed within the film externally and independently of the nanodomains.

According to some embodiments, the polymeric film layer is configured to self-adhere to the subject's tissue surface. According to some embodiments, the tissue surface may be skin, nails, lips, vaginal tissue, or buccal tissue.

According to some embodiments, the polymeric film layer is porous. According to some embodiments, the pores have a range of 10-500 micrometer. Without being bound by any theory, the nanodomains enter the pores when embedded into the film.

According to some embodiments, the film layer, having embedded therein the nanodomains, has a light transmission of at least 65%.

According to some embodiments, the polymeric film layer contains less than about 20 wt % or less than 15 wt % water. According to some embodiments, the polymeric film is essentially devoid of water.

According to some embodiments, the film may be hydrophilic.

According to some embodiments, the nanodomains within the film have an interfacial tension of substantially zero.

According to some embodiments, the nanodomains comprise at least one hydrophilic surfactant. According to some embodiments, the at least one hydrophilic surfactant has a Critical Packing Parameter (CPP) of 1.0 to 0.3.

According to some embodiments, the at least one hydrophilic surfactant is selected from: polyoxyethylene (20EO) sorbitan monolaurate, polyoxyethylene (20EO) sorbitan monopalmitate, polyoxyethylene (20EO) sorbitan monooleate, a polyoxyethylene ester of saturated or unsaturated castor oil, an ethoxylated monoglycerol ester, an ethoxylated fatty acid, an ethoxylated fatty alcohol and any combination thereof. Each possibility is a separate embodiment.

According to some embodiments, the at least one hydrophilic surfactant is selected from: polyoxyethylene, ethoxylated (20EO) sorbitan monostearate/palmitate (T60/T40), ethoxylated (20EO) sorbitan mono oleate/linoleate (T80), polyoxyethylene, ethoxylated (20EO) sorbitan monolaurate (T20), castor oil ethoxylated (20EO to 60EO); hydrogenated castor oil ethoxylated (20 to 60EO), ethoxylated (5-40 EO) monoglyceride stearate/palmitate/oleate/laurate, polyoxyl 35 castor oil, polysorbate 20 (Tween 20), polysorbate 40 (Tween 40), polysorbate 60 (Tween 60), polysorbate 80 (Tween 80), Mirj S40, Mirj S20, oleoyl macrogolglycerides, ethoxylated hydroxyl stearic acid (Solutol HS 15), a sugar ester, a polyglycerol ester, ethoxylated castor oil, a polyglycerol ester, a mono or monodi glycerol ethoxylated fatty acid (20 to 40 EU), polyoxyethylene of alkyl ethers (Brij s) and any combination thereof. Each possibility is a separate embodiment.

According to some embodiments, the at least one hydrophilic surfactant is selected from Tween 80, Tween 20, Tween 60, polyoxyl 35 castor oil (Cremophor EL®), sucrose ester monolaurate, Brij CS20, Brij C20 and any combination thereof. Each possibility is a separate embodiment.

According to some embodiments, the hydrophilic surfactant (if nonionic) has an HLB>8.

According to some embodiments, the nanodomains further comprise at least one lipophilic surfactant.

According to some embodiments, the at least one lipophilic surfactant has a CPP of about 1.0-1.3. According to some embodiments, the at least one lipophilic surfactant has an HLB<8.

According to some embodiments, the at least one lipophilic surfactant is selected from polyoxyethylene sorbitan tri oleate/stearate, sorbitan mono oleate, sorbitan mono stearate, sortbitan mono palmitate, sorbitan mono laurate, monoglyceride stearate, monoglyceride oleate, sorbitan tri stearate or tri oleate, a phospholipid, a lipophilic polyglycerol ester or polyoxyethylene ester of mono/di stearic acid or palmitic acid, lauric or oleic, polyglyceryl 3 di/monooleate, a lipophilic polyoxyehtylene alkyl ether of C10-18 chains (both saturated and mono unsaturated) and any combination thereof. Each possibility is a separate embodiment.

According to some embodiments, the at least one lipophilic surfactant comprises a phospholipid and/or a monoglyceride of a fatty acid.

According to some embodiments, the at least one phospholipid is an egg lecithin derived phospholipid, a soybean lecithin derived phospholipid, a Canola lecithin derived phospholipid, a corn lecithin derived phospholipid, a sunflower lecithin derived phospholipid, a rapeseed lecithin derived phospholipid, or phosphatidylcholine (from different sources such as rapeseed, sunflower etc.). Non-limiting examples of suitable phospholipids include Phosal, Phospholipone, Epikuron 200, LIPOID H100, LIPOID R100, LIPOID S 100, LIPOID S75, Phospholipon 90G, POPC and DOPC. Each possibility is a separate embodiment.

According to some embodiments, the nanodomains further comprise at least one short to medium chain alcohol such as ethanol, propanol, isopropyl alcohol (IPA), buthanol or any combination thereof. Each possibility is a separate embodiment.

According to some embodiments, the at least one short to medium chain poly alcohols (polyols) is selected from ethylene glycol, glycerol, polyethylene glycol, butandiol, polypropylene glycol, sorbitol, manitol, lactitol, glucose, fructose, glucoronic acid, galactose and xylitol and any combination thereof. Each possibility is a separate embodiment.

According to some embodiments, the nanodomains further comprise at least one solvent. According to some embodiments, the solvent may be any water-immiscible compound. According to some embodiments, the at least one solvent is selected from: medium-chain triglyceride (MCT), olive oil, soybean oil, castor oil, corn oil, peanuts oil, palmolein, sunflower oil, pumpkin oil, Moringa oil, cannabis oil, Canola oil, cotton seeds oil, sesame oil, grape seeds oil, avocado oil, pomegranate seeds oil, neem oil, lavender oil, peppermint oil, anise oil, ginger oil, isopropyl myristate (IPM), isopropyl palmitate (IPP), oleyl lactate, coco caprylate, hexyl laurate, benzyl alcohol, oleyl amine, oleic acid, oleyl alcohol, linoleic acid, linoleyl alcohol, ethyl oleate, hexane, heptane, nonane, decane, dodecane, D-limonene, terpenes and terpene-less (e.g. from orange, grapefruit, lemon or any other source), menthol, eucalyptol oil, capsaicin, dimethicone, cyclomethicone, tocopherols, any other essential oils or any combination thereof. Each possibility is a separate embodiment.

According to some embodiments, the at least one oil is selected from: Isopropyl Myristate (IPM), benzyl alcohol, castor oil, oleic acid, D-limonene or any combination thereof. Each possibility is a separate embodiment.

According to some embodiments, the nanodomains further comprise a co-solvent.

According to some embodiments, the co-solvent is selected from: propylene glycol (PG), glycerol, propanol, isopropanol (IPA), ethanol, polyethylene glycol (PEG), and any combination thereof. Each possibility is a separate embodiment.

According to some embodiments, the nanodomains further comprise a permeation agent (one or more). According to some embodiments, the permeation agent is selected from: diethylene glycol monoethyl ether (Transcutol®), propylene glycol, phospholipid, oleic acid, oleyl alcohol, dimethylisosorbide (DMI), benzyl alcohol, cyclodextrine, lactic acid, amine derivatives, EDTA and any combination thereof. Each possibility is a separate embodiment.

According to some embodiments, the nanodomains further comprise a membrane recognition agent. According to some embodiments, the membrane recognition agent comprises a selected phospholipid, a monoglyceride of fatty acids, a peptide, a protein or a combination thereof. Each possibility is a separate embodiment.

According to some embodiments, the drug delivery system further comprises an antioxidant (synthetic such as butylatedhydroxytoluene (BHT) or natural such as alpha-tocopherol, ascorbic acid). According to some embodiments, the drug delivery system further comprises a preservative (such as sorbate salts, benzoate salts). Non-limiting examples of suitable antioxidants include tertbutylhydroxyquinone (TBHQ), butylatedhydroxytoluene (BHT) butylated hydroxy anisole (BHA), tocopherols, ethoxylated tocopherols, tocopherol ester Propyl galate, ascorbyl palmitate, stearate and any suitable phospholipids. Each possibility is a separate embodiment.

According to some embodiments, the nanodomains in the concentrate form have a Critical Packing Parameter (CPP) or Effective Critical Packing Parameter (ECPP) of about 1.0-1.3 and in the diluted mixture prior to being casted and after re-dissolution of 0.3 to 1.0.

According to some embodiments, rewetting of the film releases/reconstructs the nanodomains therefrom.

According to some embodiments, at least a majority of the API is solubilized at the interface of the nanodomains.

According to some embodiments, the nanodomains are distributed evenly over and within the film.

According to some embodiments, the film is made of polyvinyl alcohol (PVA), carboxy methyl cellulose (CMC), methyl cellulose (MC), hydroxyethyl cellulose (HEC), hydroxypropyl cellulose (HPC), polyoxyethylene (PEO)-polyoxypropylene, (PPO)-polyoxyethylene (PEO) block copolymers (poloxamers) gelatin from bovine, gelatin and polyvinylpyrrolidone (PVP K30, PVP K90), gelatin and poloxamer, carbomer copolymers (TR1, TR2), chitosan, pectin, hyaluronic acid or any combination thereof. Each possibility is a separate embodiment.

According to some embodiments, the nanodomains comprise 2-20 wt % and/or oil soluble or oil insoluble API. According to some embodiments, at least 0.5, at least 1%, at least 2%, at least 5%, at least 10% or at least 15 wt % of the API is released from the film 1 day after being adhered to the subject's skin. Each possibility is a separate embodiment.

According to some embodiments, the nanodomains comprise a buffer. According to some embodiments, the buffer has a pH between 2 and 10. According to some embodiments, the buffer has a pH between 2 and 8. Non-limiting examples of suitable buffers include TRIS-HCL or phosphate buffer.

According to some embodiments, the polymers making up the films are crosslinked. Non-limiting examples of suitable crosslinking agents include polycarboxylic acid, citric acid, ethylenediethylamine tetra acetic acid (EDTA), malic acid, tartaric acid, succinic acid and adipic acid. Each possibility is a separate embodiment. According to some embodiments the crosslinking agent is selected from citric acid, ethylenediethylamine tetra acetic acid (EDTA), malic acid and any combination thereof. Each possibility is a separate embodiment.

According to some embodiments, the API is capable of reducing the turbidity of the film (e.g. sodium diclofenac). In other cases, the API, at least above a certain concentration, may increase the turbidity of the film.

According to some embodiments, the film comprises polymer and polyethylene glycol (PEG) and/or propylene glycol, glycerol, sorbitol and other polyolsas plasticizers. Each possibility is a separate embodiment.

According to some embodiments, the film is comprised of permeating agents. According to some embodiments, the permeating agent may be or include urea, lactic acid, propylene glycol, salicylic acid, alpha-hydroxyacids, glycolic acid, trichloroacetic acid or any combination thereof. Each possibility is a separate embodiment.

According to some embodiments, the nanodomains, when embedded in the film, weaken the hydrogen bonds of the film, as compared to a similar native, non-embedded film.

According to some embodiments, the API is positioned at the interface of the nanodomains or within the core of the nanodomain when embedded in the film.

According to some embodiments, the API travels to an interface of the nanodomain when exposed to an aqueous solution and/or a tissue surface.

According to some embodiments, there is provided a method for preparing a polymeric film having a plurality of API-containing nanodomains embedded therein, the method comprising: dissolving one or more polymers in water to form a solution; adding glycerol to the solution; adding to the solution a concentrate of nanodomains; casting the mixture on a substrate; and drying the mixture, thereby obtaining a polymeric film having a plurality of API-containing nanodomains embedded therein.

According to some embodiments, the step of forming the solution further comprises adding one or more of a buffer, a softening agent, and a water soluble plasticizer.

According to some embodiments, the step of forming the solution further comprises adding one or more of penetrating enhancers.

According to some embodiments, the step of forming the solution further comprises adding one or more of crosslinking agents. In the presence of a crosslinker, the method may further include heating the obtained film in order to allow the crosslinking of the polymers.

According to some embodiments, the polymer comprises one or more polymers, for example gelatin or gelatin together with water-soluble hydrocolloids, such as chitosan, gelatin together with poloxamer and/or PVP, cellulose derived polymer such as carboxymethyl cellulose (CMC), hydroxyethyl cellulose (HEC), hydroxypropylcellulose (HPC), methylcellulose (MC) together with Polyvinyl alcohol (PVA) and/or carbomer and/or poloxamer and/or polyvinylpyrrolidone (PVP). Each possibility is separate embodiment. Each possibility is a separate embodiment.

According to some embodiments, the step of forming the solution further comprises adding an antibacterial agent. According to some embodiments, the antibacterial agent is or contains potassium-sorbate, sodium or potassium propionate, and/or sodium benzoate and others. Each possibility is a separate embodiment.

According to some embodiments, the method further comprises a step of preparing the nanodomain concentrate.

Certain embodiments of the present disclosure may include some, all, or none of the above advantages.

One or more physical and chemical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures.

The following terms are used when describing the Figures:

a) the term ‘Native film’ refers to a casted polymer/polymers mixture film (without the nanodomains).

b) the term ‘Sol’ or mixture refers to an aqueous solution containing the film-forming polymer and other water-soluble ingredients and a concentrate of nanodomains.

c) the terms ‘Embedded film with nanodomain concentrate (no water)’, or ‘nanodomains-embedded film’ refer to the polymer film casted with empty (placebo) nanodomains.

d) the terms ‘Loaded films’ or Film embedded with API-loaded (API-solubilized) nanodomains refers to polymer films casted with nanodomains-loaded with one or more active ingredient (various loading contents).

e) the term ‘Reconstituted nanodomains’ refers to nanodomains obtained after them being released from the film into an aqueous solution.

f) the term ‘after use discharged film’ refers to film obtained after release of its API content.

g) the term ‘crosslinked film’ refers to a film made of crosslinked polymers.

FIG. 1 shows photographs (appearance) of a casted gelatin-based polymer: a) native film (without the nanodomain), b) film embedded with the placebo (empty, no API) nanodomains, and c) loaded film embedded with API containing nanodomains (Diclofenac Sodium (DCF 0.1-12% wt).

FIG. 2 shows photographs of the casted native gelatin consisting of glycerol as a plasticizer: a) native film without the nanodomains, b) film embedded with the placebo nanodomains and, c) film embedded with API-loaded nanodomains (Diclofenac Sodium (DCF) 3 wt %).

FIG. 3 shows photographs of gelatin film comprising a sorbitol as a plasticizer: a) native film, b) loaded with nanodomains without the API, and c) films embedded with the nanodomains loaded with the 3 wt % API.

FIG. 4A shows photographs of a non-compatible polymer (such as PVA): a) casted native polymers, b) casted with the embedded nanodomains (no API) and, c) casted with nanodomains loaded with the 3 wt % API.

FIG. 4B shows photographs of a casted film made of a first combination of polymers and, embedded with THD nanodomains (prepared according to Example B.4—left panel) or BJ nanodomains (prepared according to Example B.11—right panel).

FIG. 4C shows photographs of a casted film made of a second combination of polymers and embedded with (prepared according to Example B.4—left panel) or BJ nanodomains (prepared according to Example B.11—right panel).

FIG. 4D shows photographs of a casted film made of a third combination of polymers and embedded with (prepared according to Example B.4—left panel) or BJ nanodomains (prepared according to Example B.11—right panel).

FIG. 4E shows photographs of a casted film made of a fourth combination of polymers and embedded with (prepared according to Example B.4—left panel) or BJ nanodomains (prepared according to Example B.11—right panel).

FIG. 4F shows photographs of a casted film made of a fifth combination of polymers and embedded with (prepared according to Example B.4—left panel) or BJ nanodomains (prepared according to Example B.11—right panel).

FIG. 4G shows a photograph of a casted film made of a sixth combination of polymers and embedded with BJ nanodomains (prepared according to Example B.11—right panel).

FIG. 4H shows a photograph of an unsatisfactory film (prepared according to Example A.44).

FIG. 5 schematically depicts an optional setup for testing tensile strength and stress using Instron instrumentation.

FIG. 6 depicts an illustrative stress-strain curve of classical commercial films.

FIG. 7A depicts the stress-strain unique curve of native film (dark gray line), native film plus plasticizer (light gray), film embedded with empty nanodomains—(GF172 (prepared according to Example C.1)—gray line) and film embedded with sodium diclofenac (DCF)-loaded nanodomains-(GF172 stippled line).

FIG. 7B is a close-up of the stress-strain curve of the native film (prepared essentially as described in Example A.2) dark gray line) shown in FIG. 7A.

FIG. 7C is a close-up of the stress-strain curve of native film with plasticizer (gray line), film embedded with empty nanodomains—(GF172—stippled grey line) and film embedded with 3 wt % Na-DCF-loaded nanodomains—(GF172—stippled black line).

FIG. 8 is a graph showing Young's Modulus of elasticity of a film: a) embedded with 30 wt % nanodomains and, b) embedded with 30 wt % API-loaded nanodomains (3 wt % Na-DCF).

FIG. 9A shows Small Angle X-Ray Scattering (SAXS) diffractions of: a) native polymer film (stippled gray line), b) film embedded with empty nanodomains (gray line) and, c) film embedded with nanodomains loaded with 3 wt % Na-DCF (stippled black line) (from total dry film weight).

FIG. 9B shows small Angle X-Ray scattering (SAXS) diffractions of: a) film embedded with nanodomains loaded with 1 wt % terbinafine HCl, (TRB-HCl—stippled black line) and, b) film embedded with nanodomains loaded with 1 wt % cannabidiol (CBD—gray line).

FIG. 10 shows the Diffusion Coefficients (DCs) measured using Pulse Gradient Spin Echo (PGSE)-NMR, DOSY-NMR or Self Diffusion (SD) NMR of transparent reconstituted mixtures derived from: a) empty nanodomains embedded into polymer film (dark gray bars) and, b) Na-DCF (3 wt %) loaded nanodomains embedded in polymer film (light gray bars).

FIG. 11 shows Diffusion Coefficients (DCs) extracted and calculated using PGSE-NMR, DOSY-NMR or Self-Diffusion (SD) NMR of pre-casted mixtures of: a) empty nanodomains and polymer diluted with 66 wt % water (light gray bars) and, b) Na-DCF (0.25 wt %)-loaded nanodomains and polymer diluted with 66 wt % water (black bars).

FIG. 12 shows Diffusion Coefficient (DC) extracted and calculated using PGSE-NMR, DOSY-NMR or Self-Diffusion (SD) NMR of pre-casting mixtures of: a) “sol” mixture of empty nanodomains mixed with polymer (diluted with 92 wt % water) (dark gray bars) and, b) Na-DCF (0.25 wt % from the sol mixture)-loaded nanodomains and polymer diluted with 92 wt % water (light gray bars).

FIG. 13 shows Diffusion Coefficient (DC) extracted and calculated using PGSE-NMR, DOSY-NMR or Self-Diffusion (SD) NMR of: a) pre-casting mixtures of empty nanodomains and polymer diluted with 92 wt % water (dark gray bars) and, b), reconstituted mixtures derived from empty nanodomains embedded into polymer at a 92 wt % water dilution (light gray bars).

FIG. 14A shows Diffusion Coefficient (DC) extracted and calculated using PGSE-NMR, DOSY-NMR or Self-Diffusion (SD) NMR of: a) pre-casting mixtures of Na-DCF (0.25 wt %)-loaded nanodomains and polymer diluted with 92 wt % water (light gray bars) and, b), reconstituted mixtures derived from Na-DCF (0.25 wt %)-loaded nanodomains embedded into polymer at a 92 wt % water dilution (dark gray bars).

FIG. 14B shows Diffusion Coefficient (DC) extracted and calculated using PGSE-NMR, DOSY-NMR or Self-Diffusion (SD) NMR of: a) pre-casting mixtures of nanodomains loaded with 1% terbinafine HCl polymer and diluted with 92 wt % water (dark gray bars) and, b) reconstituted mixtures derived from nanodomains loaded with 1% terbinafine HCl embedded into polymer at a 92 wt % water dilution (light gray bars).

FIG. 15 depicts infra-red (IR) spectra of concentrated nanodomains (water-free) of: a) empty nanodomains (dark gray line) and, b) nanodomains loaded with 10 wt % Na-DCF (light gray line).

FIG. 16 depicts infra-red spectra of: a) native film (stippled line), b), native film+plasticizers (light gray line), c) film with empty nanodomains concentrate (dark grey line) and, d) film with nanodomain concentrate with Na-DCF (black line).

FIG. 17 depicts infra-red spectra of: a) empty nanodomains (stippled line), b) loaded nanodomains with 10 wt % Na-DCF (stippled gray line) and, c) gelatin films with Na-DCF-loaded nanodomains, different film formers were tested (gray line, polyvinylpyrrolidone (PVP) only), dark gray line (PVP and poloxamer 188), (PVP and poloxamer 407, light gray line).

FIG. 18 shows microscope images of three different films embedded with Na-DCF loaded nanodomains: a) GF171 (contains PVP only), b) GF172 (contains PVP and poloxamer 188) and, c) GF173 (contains PVP and poloxamer 407), prepared according to Example C.1.

FIG. 19A depicts illustrative graphs showing Na-DFC release from a gelatin films into receptor cells as percentage of the initial dose for GF171 (contains PVP only—dark gray bars), GF172 (contains PVP and poloxamer 188—white bars), GF173 (contains PVP and poloxamer 407—stripped bars) as compared to the commercial product Voltadol (dotted bars).

FIG. 19B depicts illustrative graphs showing Na-DFC release from a gelatin films once adhered to pig skin (in mg/cm²) as percentage of the initial dose for GF171 (contains PVP only—dark gray bars), GF172 (contains PVP and poloxamer 188—white bars), GF173 (contains PVP and poloxamer 407—stripped bars) as compared to the commercial product Voltadol (dotted bars).

FIG. 20 shows microscope images of: a) native film (magnitude×100), b) native film (magnitude×400, c), native film+plasticizers (magnitude×100), d) native film+plasticizers (magnitude×400), e) film with empty nanodomains (magnitude×100), f) film with empty nanodomains (magnitude×400), g) film with loaded nanodomains with 3 wt % Na-DCF (magnitude×100) and, h) film with loaded nanodomains with 3 wt % Na-DCF (magnitude×400).

FIG. 21A shows microscope images of a film based on PVA, CMC and poloxamer 407 with loaded nanodomains with 1 wt % terbinafine HCl (magnitude×100).

FIG. 21B shows microscope images of a film based on PVA, CMC and poloxamer 407 with loaded nanodomains with 1-wt % terbinafine HCl (magnitude×400).

FIG. 22A is a graph depicting terbinafine HCl (TRB) permeation (% from applied dose) into the receptor cell when delivered using the herein disclosed film embedded with THD-d nanodomains (prepared according to Example B.4).

FIG. 22B is a graph depicting terbinafine HCl (TRB) permeation (% from applied dose) into the receptor cell when delivered using the herein disclosed film embedded with BJ-h nanodomains (prepared according to Example B.11).

FIG. 22C is a graph depicting terbinafine HCl (TRB) permeation (% from applied dose) into the receptor cell when delivered using the herein disclosed film embedded with THD-d nanodomains (prepared according to Example B.4) and containing a permeating agent.

FIG. 23A is a graph depicting terbinafine HCl (TRB) permeation (% from applied dose) into skin when delivered using the herein disclosed film embedded with THD-d nanodomains (prepared according to Example B.4).

FIG. 23B is a graph depicting terbinafine HCl (TRB) permeation (% from applied dose) into skin when delivered using the herein disclosed film embedded with BJ-h nanodomains (prepared according to Example B.11).

FIG. 23C is a graph depicting terbinafine HCl (TRB) permeation (% from applied dose) into skin when delivered using the herein disclosed film embedded with THD-d nanodomains (prepared according to Example B.4) and containing a permeating agent.

FIG. 24A is a graph depicting terbinafine HCl (TRB) permeation (% from applied dose) into the receptor cell when delivered using the herein disclosed film embedded with THD-d nanodomains (prepared according to Example B.4) in the presence and absence of polymer cross-linking; and

FIG. 24B is a graph depicting terbinafine HCl (TRB) permeation (% from applied dose) into skin when delivered using the herein disclosed film embedded with THD-d nanodomains (prepared according to Example B.4) in the presence and absence of polymer cross-linking.

### DETAILED DESCRIPTION OF THE INVENTION

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

According to some embodiments, there is provided a novel drug delivery system comprising a soft, polymeric, optionally partially or completely cross-linked film and a plurality of nanodomains embedded within the film layer.

As used herein, the term “film” and “film layer” may be used interchangeably and refer to a thin, flexible and continuous polymeric material suitable for adherence to a subject's skin, buccal, ocular, vaginal, penis, nails and/or labial tissue. According to some embodiments, the subject may be a human or other mammal According to some embodiments, the film may be self-adhering with or without being wetted. According to some embodiments, the film may be self-adhesive e.g. due to one or more of the polymers providing adhesive properties (such as carbomer copolymers, PVP etc.).

As used herein, the terms “drug delivery system” and “drug delivery device” may be used interchangeably and refer to the film product configured for use.

According to some embodiments, the polymeric film layer may be relatively thin, i.e. have a thickness of about 200 μm-1000 μm, of about 250 μm-750 μm, of about 300μ m-600μ m, or about 300-500 μm or any other range of thicknesses within the range of 200-1000 μm. Each possibility is a separate embodiment. Advantageously, the relatively thin film ensures minimal visibility and transparency without compromising the structural integrity of the film.

According to some embodiments, the polymeric film layer may be porous (see FIG. 18). According to some embodiments, the pores may be in the range of about 100-500 micrometer, about 200-300 micrometer, about 100-200 micrometer, about 50-200 micrometer or any other range within the range of 10-500 micrometers. Each possibility is a separate embodiment.

As known in the art, films are usually in a stressed state. That is, the film “wants” to be smaller, or larger than the substrate allows it to be, hence the film is in tensile stress (film “wants” to shrink) or compressive stress (film “wants” to expand). The unit of stress is Pascal [Pa].

The herein disclosed porous films provide a three-dimensional matrix into which the empty or API-loaded nanodomains can be embedded. The porosity is an indication of the three-dimensional network of the polymeric matrix. Accordingly, the film embedded with the nanodomains has a similar stress and strain as that of the film (film former) with plasticizer, thus indicating that the nanodomains are located within the 3D network and do not significantly interfere with the elasticity of the film.

According to some embodiments, varying the thickness of the film may be used to adjust the maximal concentration of an API delivered (Cₘₐₓ) and/or the time to peak administration (Tₘₐₓ) thereby optimizing bioavailability. By way of example, utilizing a relatively thin film (e.g. below 500 microns, below 400 microns or below 375 microns) may essentially prevent irreversibly “trapping” of the API within layers of the film. As another example, using a relatively thin film (e.g. below 100 micrometer or below 50 micrometer) may ensure a short time to peak concentration (low Tₘₐₓ value). As another example, using a relatively thick film (e.g. more than 100 micrometer or more than 150 micrometer) may ensure a higher Cₘₐₓ.

According to some embodiments, the film may include at least one polymer, such as 1, 2, 3, 4 or more polymers. Each possibility is a separate embodiment.

According to some embodiments, the polymer may be a water-soluble, synthetic or semi-synthetic polymer. According to some embodiments, the polymer may be a water-soluble, hydrophilic polymer. According to some embodiments, the polymer may be a water-soluble polysaccharide or protein. According to some embodiments, the polymer may be a water-soluble anionic polysaccharide such as CMC-Na or Alginate salts. According to some embodiments, the polymer may be a biopolymer such as, but not limited to, gelatin proteins. A non-limiting example of a suitable combination of polymers include gelatin and Poloxamer; polycarboxymethylcellulose (CMC), polyvinylpyrrolidone (PVP) and Poloxamer; polyvinyl alcohol (PVA), Poloxamer and PVP, CMC, Poloxamer 407, Carbomer copolymers (TR1) and PVA. According to some embodiments, a suitable polymeric blend includes CMC, Poloxamer 407, TR1 and PVA. Each possibility is a separate embodiment.

According to some embodiments, the film polymer comprises about 5-30 wt % of the total dry weight of the drug delivery system, about 10 wt %-40 wt % of the total dry weight of the drug delivery system, about 2.0 wt %-5.0 wt % of the total dry weight of the drug delivery system or any other suitable range within the range of 2.0 wt %-30 wt % of the total dry weight of the drug delivery system. Each possibility is a separate embodiment.

According to some embodiments, the film polymer comprises about 5-30 wt % nanodomains, loaded with 0.1-20 wt % of API.

According to some embodiments, the polymeric film layer may include one or more antibacterial agents, such as, but not limited to, potassium-sorbate, sodium propionate, sodium or potassium benzonate, sulfite or EDTA salts or any combination thereof. Each possibility is a separate embodiment.

As used herein, the term “nanodomains” refer to nano-sized self-assembled delivery vehicles, i.e. nanosized monodispersed droplets that spontaneously form when solubilized/diluted at room temperature and without requiring shearing. The nanodomains are made of surfactants and small amounts of oil (e.g. about 1 wt % 10 wt % oil or about 1 wt %-5 wt %), and optionally additional components such as co-surfactants, solvents, co-solvents and other additives such as permeating agents and membrane recognition agents. The surfactants may optionally be non-ionic or zwitterionic and thus not sensitive to electrolytes and pH, however anionic and cationic surfactants are also applicable. Advantageously, the high solubilization capacity allows high drug loads in the nano-domains e.g. 0.1 to 20 wt %. Moreover, the nanodomains are advantageously, thermodynamically stable thus providing long shelf-life stability. Without being bound by any theory, the small amount of oil is important because it forces the API to reside at the interface of the nanodomains or in the core of the nanodomains thus enhancing the transport from the nanodomains into the skin, when adhered in that the nanodomains will release the API only when in touch with lipophilic cell membrane. That is, the driving force for the transport is the miscibility of the API into the membranes' lipophilic environment.

According to some embodiments, the composition of the nanodomains is specifically tailored to enable their embedding within the polymeric film without compromising the structural integrity of the nanodomains or the properties of the film. According to some embodiments, the nanodomains comprise less solvent than previously disclosed nanodomains, such as less than about 25 wt % solvent, less than about 20 wt % solvent or less than about 15 wt % solvent. Each possibility is separate embodiment. According to some embodiments, the nanodomains include about 1 wt % to about 25 wt % solvent, or about 5 wt % to about 20 wt % solvent. Each possibility is separate embodiment. According to some embodiments, the nanodomains are essentially devoid of poly-alcohol (polyols) or include only small concentrations of poly-alcohol as compared to previously disclosed nanodomains e.g. about 35 wt % poly-alcohol or less, about 25 wt % or less or about 15 wt % or less. Each possibility is separate embodiment. According to some embodiments, the nanodomains include about 1 wt % to about 35 wt % solvent, or about 5 wt % to about 25 wt % polyols. Each possibility is separate embodiment. According to some embodiments, the nanodomains comprise lower concentrations of hydrophilic and/or lipophilic surfactants as compared to previously disclosed nanodomains, e.g. within the range of about 30-60 wt % surfactant.

According to some embodiments, the nanodomains, and/or the film further include glycerol and/or sorbitol.

These self-assembled nanodomains are extremely small. According to some embodiments, the nanodomains are below 100 nm in size, below 50 nm, below 25 nm in size or below 20 nm in size. According to some embodiments, the nanodomains have a size range of 10-100 nm, 10-50 nm, 15-40 nm or 15-20 nm. As used herein, the term “size”, with regard to the nanodomains, refers to the arithmetic mean of measured droplets' diameters, wherein the diameters range±15% from the mean value. Advantageously the small size of the nanodomains ensure a clear, transparent water-like appearance when solubilized. The nanodomains may be produced in the form of a concentrate (water-free) that is fully and progressively dilutable with water as well as other aqueous solutions.

Upon dilution with water or aqueous solutions, water-in-oil (W/O) nanodomains (or nanodroplets) are formed, which are able to invert into bi-continuous mesophases in the presence of an aqueous phase, e.g. water (upon dilution). Upon further dilution, they undergo inversion (umbrella type inversion) into oil-in-water (O/W) nanodomains or droplets.

According to some embodiments, the nanodomains comprise about 10 wt %-90 wt % of the total dry weight of the drug delivery system, about 40 wt %-85 wt % of the total dry weight of the drug delivery system, about 50 wt %-80 wt % of the total dry weight of the drug delivery system, about 30 wt %-60 wt % of the total dry weight of the drug delivery system, or any other suitable range within the range of 5 wt %-60 wt % of the total dry weight of the drug delivery system. Each possibility is a separate embodiment.

According to some embodiments, the nanodomains have an interfacial tension between the oily phase and the aqueous phase of substantially zero. As used herein, the term “substantially zero” with regards to the interfacial tension may refer to an interfacial tension of below about 2 mN/m, below about 1 mN/m, below about 0.5 mN/m or below about 0.1 mN/m. Each possibility is a separate embodiment. Without being bound to any theory, the zero interfacial tension is facilitating formation of the droplets without applying shear, as demonstrated by the spontaneous curvature (Ro) of the nanodomains as well as the optimal interfacial elasticity (RE) of the nanodomains. These characteristics are obtained by careful tailoring of the nature, composition and amounts of the surfactant(s), co-surfactants (s), solvent(s) and/or co-solvent(s) of the oil phase.

According to some embodiments, the nanodomains include at least one hydrophilic surfactant. According to some embodiments, at least one hydrophilic surfactant may refer to a single hydrophilic surfactant or to a mixture of 2, 3, 4 or more hydrophilic surfactants (e.g. ethoxylated sorbitan mono oleate (Tween 80) and ethoxylated castor oil (ECO 35), or hydrogenated ethoxylated castor oil (HECO 40) or hydrophilic ethoxylated alkyl ethers of fatty alcohols. Each possibility is a separate embodiment.

According to some embodiments, the at least one hydrophilic surfactant is a medium or long-chain lipophilic tail (C12-C24, saturated or non-saturated, oil soluble) and a large hydrophilic head (EO 40-20, water soluble or hydratable).

According to some embodiments, the at least one hydrophilic surfactant has a Critical Packing Parameter (CPP) in the proximity of 0.3 but not higher than 1.0, wherein the CPP is defined by the length of the tail (l), the surface area of the head group (a) and the volume of the surfactant(v) i.e. CPP=a·l/v

According to some embodiments, the at least one hydrophilic surfactant is selected from: polyoxyethylene sorbitan monolaurate, polyoxyethylene sorbitan monopalmitate, polyoxyethylene sorbitan monooleate, polyoxyethylene sorbitan monostearate, a polyoxyethylene ester of saturated or unsaturated castor oil, an ethoxylated monoglycerol ester, an ethoxylated fatty acid esters, ethoxylated alkyl ethers of fatty alcohols, polyglycerol esters of fatty acids, sucrose ester of fatty acids and any combination thereof. Each possibility is a separate embodiment.

According to some embodiments, the at least one hydrophilic surfactant may have a hydrophilic lipophilic balance of above 10 (HBL>10). According to some embodiments, the at least one hydrophilic surfactant is selected from: polyoxyethylene, ethoxylated (20EO) sorbitan monolaurate (T20), ethoxylated (20EO) sorbitan monostearate/palmitate (T60/T40), ethoxylated (20EO) sorbitan mono oleate/linoleate (T80), castor oil ethoxylated (20EO to 60EO); hydrogenated castor oil ethoxylated (20 to 60EO), ethoxylated (5-40 EO) monoglyceride stearate/palmitate, polyoxyl 35 castor oil, polysorbate 20 (Tween 20), polysorbate 40 (Tween 40), polysorbate 60 (Tween 60), polysorbate 80 (Tween 80), Mirj S40, Mirj S20, oleoyl macrogolglycerides, ethoxylated hydroxyl stearic acid (Solutol HS 15), a sugar esters such as sucrose mono oleate, mono laurate, monoplamitate and monostearate, a polyglycerol ester of oleic acid and caprylic/capric acids, ethoxylated castor oil, a polyglycerol ester, a mono or di glycerol ethoxylated fatty acid (20 to 40 EO), ethoxylated (EO 10-25) alkyl ethers (hydrophobic chain of C8-18 including C18:0 and C18:1) and any combination thereof. Each possibility is a separate embodiment.

According to some embodiments, the at least one hydrophilic surfactant is selected from sorbitan mono oleate/linoleate Tween 80, polyoxyl 35 castor oil ethoxylated (35EO), Hydrogenated castor oil ethoxylated (40-60), sucrose ester of monolaurate mono-oleate, monopalmitate, mono-myristate and any combination thereof. Each possibility is a separate embodiment. According to some embodiments, the nanodomains further comprise at least one lipophilic surfactant, also referred to herein as a co-surfactant. According to some embodiments, the lipophilic surfactant has a hydrophilic lipophilic balance of below 10 (HLB<10). According to some embodiments, at least one lipophilic surfactant may refer to a single lipophilic surfactant or to a mixture of 2, 3, 4 or more lipophilic surfactants. Each possibility is a separate embodiment.

As used herein, the term co-surfactant may encompass any agent, different from the lipophilic surfactant, which is capable (together with the hydrophilic surfactant) of lowering the interfacial tension between the oil phase and the aqueous phase to almost zero (or zero) allowing for the formation of a homogeneous oily mixture, thereby providing for continuous and progressive dilution of the nanodomains within an aqueous diluent, as well as assisting in maintaining the integrity of the nano-domains.

According to some embodiments, the at least one lipophilic surfactant has a relatively short to long-chain lipophilic tail (C8-C24) and a small hydrophilic head. (EO5-EO15).

According to some embodiments, the at least one lipophilic surfactant has a CPP of about 1.0 to 1.3. That is, according to some embodiments, the nanodomains may include or consist of a hydrophilic surfactant (CPP 0.3) and lipophilic surfactant with CPP of 1.0 to 1.3.

According to some embodiments, the at least one lipophilic surfactant is selected from sorbitan, monoglyceride stearate, sorbitan monooleate, sorbitan monolaurate, sorbitan monopalmitate, sorbitan monostearate, sorbitan tri stearate or tri oleate, a phospholipid, a polyglycerol ester of stearic acid or palmitic acid or lauric or oleic or recinoleic, ethoxylated (EO 2-10) alkyl ethers of lauryl alcohol or cetyl alcohol, or stearoyl alcohol or cetostearyl alcohol or oleyl alcohol and any other suitable lipophilic surfactant or combination of surfactants. Each possibility is a separate embodiment.

According to some embodiments, the at least one lipophilic surfactant comprises a phospholipid and/or a monoglyceride of a fatty acid. According to some embodiments, the at least one phospholipid is selected from egg lecithin, soybean lecithin, canola lecithin, corn lecithin, sunflower lecithin, rapeseed lecithin, hydrogenated lecithin, phosphatidylcholine, Phosal, phospholipone, Epikuron 200, LIPOID H100, LIPOID R100, LIPOID S 100, LIPOID S75, POPC, DOPC, PHOSPHOLIPON 90G or PHOSPHOLPON 90H, and any combination thereof. Each possibility is a separate embodiment.

Additionally or alternatively, the nanodomains may further include at least one short to medium chain alcohol. According to some embodiments, at least one short to medium chain alcohol may refer to a short to medium chain alcohol or to a mixture of 2, 3, 4 or more short to medium chain alcohols. Each possibility is a separate embodiment.

That is, according to some embodiments, the nanodomains may include or consist of a hydrophilic surfactant Critical Packing Parameter (CPP) of 0.3 to 1.0 and short, or medium chain alcohol, such which together form an Effective Critical Packing Parameter (ECPP) of 0.3 to 1.0 depending on the chemical composition of the nanodomains and the amount of water.

According to some embodiments, the at least one short to medium chain alcohol are polyols, selected from ethylene glycol, glycerol, polyethylene glycol, polypropylene glycol (or polyoxyethylene), sorbitol, lycopene, lactitol, and xylitol and other polyols such as glucose, fructose, galactose and any combination thereof. Each possibility is a separate embodiment.

Additionally, or alternatively, the nanodomains may further include at least one solvent. According to some embodiments, at least one solvent may refer to organic solvent or to a mixture of 2, 3, 4 or more organic solvents. Each possibility is a separate embodiment.

According to some embodiments, the nanodomains further comprise at least one solvent. According to some embodiments, the at least one solvent is selected from: medium-chain triglyceride (MCT), olive oil, soybean oil, corn oil, peanuts oil, palmolein, sunflower oil, pumpkin oil, moringa oil, cannabis oil, canola oil, cotton seeds oil, sesame oil, grape seeds oil, avocado oil, pomegranate seeds oil, neem oil, lavender oil, peppermint oil, anise oil, ginger oil, isopropyl myristate (IPM), isopropyl palmitate (IPP), oleyl lactate, coco caprylate, hexyl laurate, benzyl alcohol, oleyl amine, oleic acid, oleyl alcohol, linoleic acid, linoleyl alcohol, ethyl oleate, hexane, heptane, nonane, decane, dodecane, D-limonene, terpenes and terpene-less (from orange, grapefruit, lemon or from any source, menthol, eucalyptol oil, capsaicin, dimethicone, cyclomethicone or any combination thereof.

According to some embodiments, the at least one oil is selected from: Isopropyl Myristate (IPM), benzyl alcohol, castor oil, D-limonene, oleyl alcohol, oleic acid, or any combination thereof. Each possibility is a separate embodiment.

Additionally or alternatively, the nanodomains may further include a co-solvent. According to some embodiments, at least one co-solvent may refer to a co-solvent or to a mixture of 2, 3, 4 or more co-solvents. Each possibility is a separate embodiment.

According to some embodiments, the co-solvent is selected from: propylene glycol (PG), glycerol, propanol, isopropanol (IPA), ethanol, polyethylene glycol (PEG) and any combination thereof. Each possibility is a separate embodiment.

According to some embodiments, the nanodomains may further include a permeation (or penetration) agent or a mixture of two or more permeation agents. As used herein, the term “permeation agent” may refer to any agent capable of enhancing the permeation of the nanodomains APIs through the subject’ skin and thus increase the effectiveness of the delivery. According to some embodiments, the permeation agent is selected from: diethylene glycol, propylene glycol, monoethyl ether (Transcutol®), 1,3-Dimethyl-2-imidazolidinone (DMI), a phospholipid, oleic acid, oleyl alcohol olive oil, sesame oil, and any combination thereof. Each possibility is a separate embodiment.

According to some embodiments, the nanodomains may further include a “membrane recognition agent” or a mixture of two or more “membrane recognition agents”. As used herein, the term “membrane recognition agent” may refer to any agent having selective affinity to cell membranes or skin surfaces and thus for targeted delivery, e.g. to cells in general or to particular subset of cells, e.g. anionic cell membrane surfaces of cancer cells as opposed to near-neutral membrane surfaces of healthy mammalian cells.

According to some embodiments, the membrane recognition agent may be any phospholipid, a monoglyceride of fatty acids, membrane recognition peptides or proteins or a combination thereof.

According to some embodiments, the nanodomains may further include an antioxidant (AO), a preservative and/or a viscosity agent. Each possibility is a separate embodiment. According to some embodiments, the antioxidants may be a naturally occurring antioxidant or synthetic antioxidant. Examples of antioxidants can be compounds which react with free radicals (e.g. tocopherol or its derivatives, i.e. tocopherol acetate), reducing agents or antioxidants that can lower the redox potential of the API and prolong its stability. Examples of natural antioxidants can be molecules such as ascorbic acid, ascorbyl palmitate or other ascorbic acid derivatives. Examples of synthetic antioxidants can be molecules such as BHA, BHT, TBHQ, propyl lycopen and others. Combinations of antioxidants may also be synergists, which enhance the antioxidants activity.

According to some embodiments, the nanodomains may include excipients capable of inhibiting bacterial and/or fungal growth, such as those selected from EDTA (disodium ethylenediametetraacetate), sodium metabisulfite, acetic acid, sodium propionate, sorbic acid and its potassium or sodium salt, lycopene, pentetate, benzyl alcohol, benzalkonium chloride, and sodium benzoate and other antimicrobial agents known to the art. Each possibility is a separate embodiment.

As used herein, the term “active pharmaceutical ingredient (API)” refers to any active ingredient (AI) such as a pharmaceutical drug, a nutraceutical (i.e. any bioactive derived from plants, or animal, or fish, or sea food sources or synthetic (flowers, seeds, fruits, leaves, roots, bark, etc.) sources with health benefits in addition to the basic nutritional value, an active ingredient for cosmetic purposes (cosmeceuticals) or any other active or bioactive substance. According to some embodiments, the API may be compound(s) water and/or oil insoluble, i.e. having a water and oil solubility of below 0.5 wt %. According to some embodiments, the API may be partially lipophilic (some miscibility in “oil” phases) or lipophilic (soluble in organic solvents or “oils).

According to some embodiments, the API may be a nutraceutical. Non-limiting examples of suitable nutraceuticals include astaxanthin, zeaxanthin, lycopene. Lutein, beta carotene, flucoxanthin, canthaxanthin and other carotenoids, rapeseed oil, pomegranate oil, pumpkin oil. Morula oil, cannabinoids (CBD, THC and other cannabinoids), omega fatty acids, theacrine, CoQ10, Bowsella, vitamin D3, tocopherols, and curcumin According to some embodiments, the API may be a pharmaceutical agent such as, but not limited to, an analgesic (e.g. diclofenac sodium, ibuprofen and others) antifungal agent (e.g. terbinafine, ketoconazole, colistib, daptomycin, teicoplanin, taltirelin, thymopentin, vancomycin and others), a cannabinoid (e.g. cannabidiol (CBD) and Δ9-tetrahydrocannabinol (THC)), an anesthetic (e.g. propofol and lidocaine), antibiotics (e g minocycline, desoxycycline, colistin, daptomycin, teicoplanin, taltirelin, thymopentin, vancomycin), an eicosanoid (e.g. alprostadil), an anti-viral agent (e.g. Chloroqunine, Hydroxychloroquinine, Remdesivir, Lopinavir, Titonavir, Kaltera and others), an anti-bacterial drugs (e.g. Colomicin, Cubimicin, Targicid, Cerrsdit, Ziconatide and others), a hormone, an anti-cancer drugs (e.g. Lucentis and Avastin) a biopolymer (e.g. hyaluronic acid, insulin, cyclosporine, calcitonin, a protein or peptide, an RNA and any combination thereof. Each possibility is a separate embodiment.

According to some embodiments, the nanodomains may be loaded with an effective amount of API. The term “effective amount” for purposes herein may be determined by considerations known in the art. The effective amount is typically determined in appropriately designed clinical or preclinical trials (dose range studies) and the person versed in the art will know how to properly conduct such trials in order to determine the effective amount. As generally known, the effective amount depends on a variety of factors including the distribution profile within the body, a variety of pharmacological parameters such as half-life in the body, on undesired side effects, if any, on factors such as age and gender, and others.

According to some embodiments, the API is solubilized within the core and/or at the interface of the nanodomains. According to some embodiments, some groups of the API may be anchored at the interface awhile other groups of the API are dangling in the aqueous phase. According to some embodiments, at least some or a majority of the API is solubilized at the interface of the nanodomains. According to some embodiments, the remainder of the API may be contained within the core of the nanodomains or in the aqueous continuous phase.

According to some embodiments, the nanodomains comprise about 0.1%-30% w/w of API, about 0.1%-25% w/w of API, or about 2%-20% w/w of API or any other range within the range of 0.5%-30% API. Each possibility is a separate embodiment.

According to some embodiments, the film is configured to controllably release the API and/or the nanodomains upon being adhered, or attached to the subject's skin, buccal tissue, labial tissue, penis skin, nails or other. According to some embodiments, the film is configured to controllably release the API and/or the nanodomains such that at least 15% of the API is released from the film 1 to 3 days after being adhered to the subject's skin.

According to some embodiments, the film further includes API (the same or a different API), directly incorporated into the film, i.e. without being loaded on nanodomains.

According to some embodiments, the delivery system, i.e. the film, having embedded therein the nanodomains, is transparent or translucent, or slightly opaque. According to some embodiments, the film, having embedded therein the nanodomains, has a light transmission of at least at least about 70 wt %, at least about 80 wt %, or at least about 90%. Each possibility is a separate embodiment.

According to some embodiments, the nanodomains maintain their structural integrity within the film when stored at room temperature for at least 6-12 months, such that release of the API from the film during storage is essentially prevented. As used herein” the term “maintain structural integrity” refers to the ability to identify nanodomains upon dissolution of the film within which they are embedded. According to some embodiments, the nanodomains' structure is maintained when embedded within the film. Alternatively, the nanodomains are reconstructed (reconstituted) upon the dissolution/wetting of the film and/or upon their release from the film as a result of adherence to the skin, buccal, penis skin, nails or labial tissue. In any event, the structure of the nanodomains is sufficiently preserved to prevent essentially any or only residual release of the API (i.e. less than 0.2 wt % or 0.1 wt %, or 0.05 wt % of its initial concentration, each possibility being a separate embodiment) during storage of the delivery system and/or prior to adherence to a subject's skin, buccal or labial tissue.

According to some embodiments, the drug delivery system contains less than about 15 wt % water. According to some embodiments, the drug delivery system is essentially devoid of water or contains only a residual amount of water i.e. less than about 1 wt % of its total weight, less than about 0.5 wt % of its total weight or less than about 0.1 wt % of its total weight, each possibility being a separate embodiment.

According to some embodiments, the nanodomains maintain an interfacial tension of substantially zero when embedded within the film.

According to some embodiments, the nanodomains are distributed evenly/homogenously over and within the film. As used herein, the term “evenly distributed” and homogenously distributed” may be used interchangeably and may refer to the nanodomain being dispersed in an essentially even concentration (e.g. +/−5% variations in the concentration) over the entire film and/or over the part of the film designated to receive the nanodomains, e.g. the entire film apart from its border, wherein the border of the film refers to a “frame” being 0.5 mm-5 mm in width, such as less than five 5 mm, less than 3 mm, less than 2 mm or less than 1 mm Each possibility is a separate embodiment.

According to some embodiments, the film, having embedded therein the nanodomains, has a tensile strength of about 1 kPa or below, of about 0.5 kPa or below or of about 0.1 kPa or below, each possibility being a separate embodiment.

According to some embodiments, the film or film layer has a Young's Modulus elasticity of about 5 MPa or below, 3 MPa or below and 1.5 MPa or below. According to some embodiments, the film or film layer has a Young's Modulus elasticity in a range of about 0.1 Kpa-5 MPa to about. According to some embodiments, the film or film layer has a Young's Modulus elasticity in a range of about 0.1 KPa-3 MPa. According to some embodiments, the film or film layer has a Young's Modulus elasticity in a range of about 0.1 MPa-1.5 MPa. According to some embodiments, the film has a Young's Modulus elasticity in a range of about 0.1 MPa-1.0 MPa. According to some embodiments, the film or film layer has a tensile strength at breaking point of about 2 MPa or below. According to some embodiments, the film or film layer has a tensile strength at breaking point in a range of about 1.5 MPa or below. According to some embodiments, the film has a tensile strength at breaking point in a range of about 0.2 Kpa-1.5 MPa. According to some embodiments, the film has a tensile strength at breaking point in a range of about 0.2 Kpa-1 MPa.

According to some embodiments, there is provided a method for preparing the polymeric film having a plurality of API-containing nanodomains embedded therein, as essentially described herein.

According to some embodiments, the method includes dissolving one or more polymers in water to form a solution; adding to the solution a concentrate (oil-phase) of optionally API-containing nanodomains and plasticizers such as glycerol, casting the mixture on a substrate; and drying the mixture, thereby obtaining a polymeric film having a plurality of API-containing nanodomains embedded therein. A further elaboration of the method of preparation can be found in the herein below experimental section.

According to some embodiments, forming the solution further comprises adding one or more of a buffer, such as phosphate buffer, acetate buffer etc., a softening agents and a water soluble plasticizer such as glycerol.

According to some embodiments, forming the solution further comprises adding an antibacterial agent.

According to some embodiments, the method further comprises a step of preparing the concentrate of API-containing nanodomains.

The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between. Accordingly, the description of a range should be considered to have specifically disclosed all the possible sub-ranges as well as individual numerical values within that range.

As used herein, the term “about” is meant to encompass deviation of ±10% from the specifically mentioned value of a parameter, such as temperature, pressure, concentration, etc.

The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

According to some embodiments, the film or film layer includes a permeating agent.

According to some embodiments, the polymers of the film or film layer may be crosslinked. According to some embodiments, the crosslinking is feasible when a crosslinker (a polycarboxylic acid) is added to the film forming solution following heating to at least at least 40° C., at least 50° C., at least 60° C. or at least 70° C. of the resulting film. Each possibility is a separate embodiment. The crosslinking may be complete or partial. Without being bound by any theory, the crosslinking reaction may be essential for the swelling of the film upon contact with an aqueous solution. Crosslinked films may or may not keep their integrity in the presence of water depending on the degree of crosslinking.

According to some embodiments, the crosslinked films are capable of absorbing water up to about 3-fold, about 5-fold, about 10-fold or about 15-fold the film's weight. Each possibility is a separate embodiment.

Non-limiting examples of suitable crosslinking agents include: citric acid, ethylenediethylamine tetra acetic acid (EDTA), malic acid, tartaric acid, succinic acid and adipic acid.

### EXAMPLES

### Example 1: Polymer Screening Experiments

The goal_of this stage was to find suitable porous and thin film-former polymers having the ability to form films with the capability of embedment (adsorption, incorporation) of the designed liquid nanodomains. The films can serve as semi-solid reservoirs for enhanced delivery of the nanodomains from the film, optionally followed by the release of the API's from the nanodomains in a slow and controlled manner.

Polymer Solution Preparation:

Several different water soluble polymers, including Gelatin from bovine, Gelatin from porcine skin, Gelatin from fish, Polyvinylpyrrolidone (PVP, Kollidon 90, Kollidon 30), copolymer of 1-vinyl-2-pyrrolidone, vinyl acetate (Copovidone, Kollidon VA-64), polycarboxymethylcellulose (CMC medium, CMC high), hydroxyl ethyl cellulose, methyl cellulose, poloxamer (188, 407), polyvinyl alcohol (PVA), carbomers of homopolymers and copolymers (Carbopol 971, Pemulen TR1, Pemule TR2) were tested. Polymer solution (0.1-7.2 wt %) were prepared in water by adding the calculated amount of polymer to a preheated aqueous solution, while stirring/mixing until complete dissolution of the polymer. To obtain a polymer solution (“sols”) addition of plasticizer is needed, e.g. glycerol and/or sorbitol.

Example A.1: 1.8 gr of gelatin from bovine were added to 100 gr of water and mechanically (severe mixing) mixed at 40° C. for up to 1 h to obtain a 1.8 wt % of gelatin aqueous solution. 4 gr of glycerol is mixed with the gelatin solution for 15 min to obtain sol.
Example A.2: 1.8 gr of gelatin from bovine and 0.2 gr PVP-Kollidon 90 were added to 100 gr of water and mechanically (severe mixing) mixed at 40° C. for up to 1 h to obtain a 2 wt % of polymer aqueous solution. 1.5 gr of sorbitol is mixed with the gelatin solution for 15 min to obtain sol.
Example A.3: 2.0 gr of PVA and 2.0 gr CMC-medium molecular weight were added to 74.5 gr of water and mechanically (severe mixing) mixed at 40° C. for up to 1 h to obtain a 5.1 wt % of polymer aqueous solution. 10 gr of glycerol is mixed with the polymers solution for 15 min to obtain sol.
Example A.4: 2.0 gr of CMC-medium molecular weight, 0.5 g Poloxamer 407 and 0.5 g PVP-K30 were added to 82 gr of water and mechanically (severe mixing) mixed at 40° C. for up to 1 h to obtain a 3.5 wt % of polymer aqueous solution. 10 gr of glycerol is mixed with the polymers solution for 15 min to obtain sol.
Example A.5: 2.0 gr of CMC-medium molecular weight, 0.7 g Poloxamer 407 and 0.3 g Pemulen TR1 were added to 82 gr of water and mechanically (severe mixing) mixed at 40° C. for up to 1 h to obtain a 3.5 wt % of polymer aqueous solution. 10 gr of glycerol is mixed with the polymers solution for 15 min to obtain sol.
Example A.6: 5.0 gr of HEC and 1.0 g PVP K30 were added to 79 gr of water and mechanically (severe mixing) mixed at 40° C. for up to 1 h to obtain a 7.1 wt % of polymer aqueous solution. 10 gr of glycerol is mixed with the polymers solution for 15 min to obtain sol.
Example A.7: 1.0 gr of PVA, 2.0 g MC and 0.5 g Poloxamer 407 were added to 81.5 gr of water and mechanically (severe mixing) mixed at 40° C. for up to 1 h to obtain a 4.1 wt % of polymer aqueous solution. 10 gr of glycerol is mixed with the polymers solution for 15 min to obtain sol.
Example A.8: 4.0 gr of gelatin and 2.0 g PVA were added to 74.5 gr of water and mechanically (severe mixing) mixed at 40° C. for up to 1 h to obtain a 7.5 wt % of polymer aqueous solution. 12 gr of glycerol is mixed with the polymers solution for 15 min to obtain sol.
Example A.9: 4.0 gr of gelatin, 2.0 g PVA and 0.5 g Poloxamer 407 were added to 74 gr of water and mechanically (severe mixing) mixed at 40° C. for up to 1 h to obtain a 8.1 wt % of polymer aqueous solution. 12 gr of glycerol is mixed with the polymers solution for 15 min to obtain sol.
Example A.10: 2.0 gr of CMC medium molecular weight, 2.0 g PVA and 0.5 g Poloxamer 407 were added to 76 gr of water and mechanically (severe mixing) mixed at 40° C. for up to 1 h to obtain a 5.6 wt % of polymer aqueous solution. 12 gr of glycerol is mixed with the polymers solution for 15 min to obtain sol.
Example A.11: 2.0 gr of PVA and 2.0 gr HEC were added to 81 gr of water and mechanically (severe mixing) mixed at 40° C. for up to 1 h to obtain a 4.7 wt % of polymer aqueous solution. 10 gr of glycerol is mixed with the polymers solution for 15 min to obtain sol.
Example A.12: 2.0 gr of PVA and 2.0 gr PVP K30 were added to 81 gr of water and mechanically (severe mixing) mixed at 40° C. for up to 1 h to obtain a 4.7 wt % of polymer aqueous solution. 10 gr of glycerol is mixed with the polymers solution for 15 min to obtain sol.
Example A.13: 2.0 gr of PVA, 1.0 gr PVP K30 and 1.0 gr poloxamer 407 were added to 81 gr of water and mechanically (severe mixing) mixed at 40° C. for up to 1 h to obtain a 4.7 wt % of polymer aqueous solution. 10 gr of glycerol is mixed with the polymers solution for 15 min to obtain sol.
Example A.14: 2.0 gr of PVA and 2.0 gr MC were added to 81 gr of water and mechanically (severe mixing) mixed at 40° C. for up to 1 h to obtain a 4.7 wt % of polymer aqueous solution. 10 gr of glycerol is mixed with the polymers solution for 15 min to obtain sol.
Example A.15: 2.0 gr of PVA, 1.0 gr MC and 0.5 gr poloxamer 407 were added to 81.5 gr of water and mechanically (severe mixing) mixed at 40° C. for up to 1 h to obtain a 4.1 wt % of polymer aqueous solution. 10 gr of glycerol is mixed with the polymers solution for 15 min to obtain sol.
Example A.16: 2.0 gr of CMC medium molecular weight and 1.0 gr PVP K30 were added to 82 gr of water and mechanically (severe mixing) mixed at 40° C. for up to 1 h to obtain a 3.5 wt % of polymer aqueous solution. 10 gr of glycerol is mixed with the polymers solution for 15 min to obtain sol.
Example A.17: 2.0 gr of CMC medium molecular weight and 2.0 gr HEC were added to 81 gr of water and mechanically (severe mixing) mixed at 40° C. for up to 1 h to obtain a 4.7 wt % of polymer aqueous solution. 10 gr of glycerol is mixed with the polymers solution for 15 min to obtain sol.
Example A.18: 2.0 gr of CMC-medium molecular weight, 0.7 g Poloxamer 407 and 0.3 g Pemulen TR2 were added to 82 gr of water and mechanically (severe mixing) mixed at 40° C. for up to 1 h to obtain a 3.5 wt % of polymer aqueous solution. 10 gr of glycerol is mixed with the polymers solution for 15 min to obtain sol.
Example A.19: 2.0 gr of CMC-medium molecular weight, 0.5 g Poloxamer 407 and 0.5 gr Carbopol 971 were added to 82 gr of water and mechanically (severe mixing) mixed at 40° C. for up to 1 h to obtain a 3.5 wt % of polymer aqueous solution. 10 gr of glycerol is mixed with the polymers solution for 15 min to obtain sol.
Example A.20: 5.0 gr of HEC were added to 80 gr of water and mechanically (severe mixing) mixed at 40° C. for up to 1 h to obtain a 5.9 wt % of polymer aqueous solution. 10 gr of glycerol is mixed with the polymers solution for 15 min to obtain sol.
Example A.21: 2.0 gr of MC and 2.0 gr PVP K30 were added to 81 gr of water and mechanically (severe mixing) mixed at 40° C. for up to 1 h to obtain a 4.7 wt % of polymer aqueous solution. 10 gr of glycerol is mixed with the polymers solution for 15 min to obtain sol.
Example A.22: 5.0 gr of gelatin, 0.5 g PVP K30 and 0.5 g Poloxamer 407 were added to 75 gr of water and mechanically (severe mixing) mixed at 40° C. for up to 1 h to obtain a 7.4 wt % of polymer aqueous solution. 9 gr of glycerol is mixed with the polymers solution for 15 min to obtain sol.
Example A.23: 1.0 gr of PVA, 1.0 g CMC medium molecular weight and 0.25 g Poloxamer 407 were added to 87.5 gr of water and mechanically (severe mixing) mixed at 40° C. for up to 1 h to obtain a 2.5 wt % of polymer aqueous solution. 5.25 gr of glycerol is mixed with the polymers solution for 15 min to obtain sol.
Example A.24: 1.5 gr of CMC medium molecular weight, 0.25 g PVP K30 and 0.25 g Poloxamer 407 were added to 90.0 gr of water and mechanically (severe mixing) mixed at 40° C. for up to 1 h to obtain a 2.5 wt % of polymer aqueous solution. 4.0 gr of glycerol is mixed with the polymers solution for 15 min to obtain sol.
Example A.25: 1.0 gr of CMC medium molecular weight, 0.5 gr HEC, 0.25 g PVP K30 and 0.25 g Poloxamer 407 were added to 90.0 gr of water and mechanically (severe mixing) mixed at 40° C. for up to 1 h to obtain a 2.2 wt % of polymer aqueous solution. 4.0 gr of polymers is mixed with the gelatin solution for 15 min to obtain sol.
Example A.26: 1.25 gr of CMC medium molecular weight, 0.525 g PVP K30 and 0.225 g Pemulene TR2 were added to 90.0 gr of water and mechanically (severe mixing) mixed at 40° C. for up to 1 h to obtain a 2.2 wt % of polymer aqueous solution. 4.0 gr of glycerol is mixed with the polymers solution for 15 min to obtain sol.
Example A.27: 5.0 gr of gelatin, 0.5 g PVP K30 and 0.5 g poloxamer 407 were added to 75.0 gr of water and mechanically (severe mixing) mixed at 40° C. for up to 1 h to obtain a 7.4 wt % of polymer aqueous solution. 11.5 gr of glycerol is mixed with the polymers solution for 15 min to obtain sol.
Example A.28: 1.0 gr of CMC medium molecular weight, 1.0 PVA and 0.25 g poloxamer 407 were added to 87.5 gr of water and mechanically (severe mixing) mixed at 40° C. for up to 1 h to obtain a 2.5 wt % of polymer aqueous solution. 6.5 gr of glycerol is mixed with the polymers solution for 15 min to obtain sol.
Example A.29: 1.5 gr of CMC medium molecular weight, 0.25 g PVP K30 and 0.25 g Poloxamer 407 were added to 92.0 gr of water and mechanically (severe mixing) mixed at 40° C. for up to 1 h to obtain a 2.1 wt % of polymer aqueous solution. 3.6 gr of glycerol is mixed with the polymers solution for 15 min to obtain sol.
Example A.30: 1.0 gr of CMC medium molecular weight, 0.5 gr HEC, 0.25 g PVP K30 and 0.25 g Poloxamer 407 were added to 92.5 gr of water and mechanically (severe mixing) mixed at 40° C. for up to 1 h to obtain a 2.1 wt % of polymer aqueous solution. 3.25 gr of glycerol is mixed with the polymers solution for 15 min to obtain sol.
Example A.31: 1.25 gr of CMC medium molecular weight, 0.525 g PVP K30 and 0.225 g Pemulene TR2 were added to 92.5 gr of water and mechanically (severe mixing) mixed at 40° C. for up to 1 h to obtain a 2.1 wt % of polymer aqueous solution. 3.25 gr of glycerol is mixed with the polymers solution for 15 min to obtain sol.
Example A.32: 1.0 gr of CMC medium molecular weight, 1.0 PVA and 0.25 g poloxamer 407 were added to 88.0 gr of water and mechanically (severe mixing) mixed at 40° C. for up to 1 h to obtain a 2.5 wt % of polymer aqueous solution. 5.75 gr of glycerol is mixed with the polymers solution for 15 min to obtain sol.
Example A.33: 0.75 gr of CMC medium molecular weight, 1.25 PVA and 0.25 g poloxamer 407 were added to 88.0 gr of water and mechanically (severe mixing) mixed at 40° C. for up to 1 h to obtain a 2.5 wt % of polymer aqueous solution. 6.0 gr of glycerol is mixed with the polymers solution for 15 min to obtain sol.
Example A.34: 2.0 gr of pectin, 0.2 PVP K30 and 0.2 g poloxamer 407 were added to 77.6 gr of water and mechanically (severe mixing) mixed at 40° C. for up to 1 h to obtain a 3.0 wt % of polymer aqueous solution. 10.0 gr of glycerol is mixed with the polymers solution for 15 min to obtain sol.
Example A.35: 1.75 gr of HPC, 1.0 PVA and 0.25 g poloxamer 407 were added to 90.0 gr of water and mechanically (severe mixing) mixed at 40° C. for up to 1 h to obtain a 3.2 wt % of polymer aqueous solution. 4.0 gr of glycerol is mixed with the polymers solution for 15 min to obtain sol.
Example A.36: 1.2 gr of CMC medium molecular weight, 1.5 PVA and 0.25 g poloxamer 407 were added to 84.05 gr of water and mechanically (severe mixing) mixed at 40° C. for up to 1 h to obtain a 3.4 wt % of polymer aqueous solution. 6.0 gr of glycerol is mixed with the polymers solution for 15 min to obtain sol.
Example A.37: 3.0 gr of pectin, 0.5 PVP K30 and 0.2 g poloxamer 407 were added to 69.3 gr of water and mechanically (severe mixing) mixed at 40° C. for up to 1 h to obtain a 5.1 wt % of polymer aqueous solution. 12.0 gr of glycerol is mixed with the polymers solution for 15 min to obtain sol.
Example A.38: 1.0 gr of CMC high molecular weight, 1.0 PVA and 0.25 g poloxamer 407 were added to 88.0 gr of water and mechanically (severe mixing) mixed at 40° C. for up to 1 h to obtain a 2.5 wt % of polymer aqueous solution. 6.0 gr of glycerol is mixed with the polymers solution for 15 min to obtain sol.
Example A.39: 0.75 gr of CMC high molecular weight, 1.0 PVA and 0.25 g poloxamer 407 were added to 89.5 gr of water and mechanically (severe mixing) mixed at 40° C. for up to 1 h to obtain a 2.2 wt % of polymer aqueous solution. 4.5 gr of glycerol is mixed with the polymers solution for 15 min to obtain sol.
Example A.40: 1.0 gr of CMC medium molecular weight, 0.75 Pemulene TR1 and 0.25 g poloxamer 407 were added to 88.0 gr of water and mechanically (severe mixing) mixed at 40° C. for up to 1 h to obtain a 2.2 wt % of polymer aqueous solution. 6.0 gr of glycerol is mixed with the polymers solution for 15 min to obtain sol.
Example A.41: 1.0 gr of CMC medium molecular weight, 1.0 PVA and 0.25 g poloxamer 188 were added to 87.5 gr of water and mechanically (severe mixing) mixed at 40° C. for up to 1 h to obtain a 2.5 wt % of polymer aqueous solution. 6.5 gr of glycerol is mixed with the polymers solution for 15 min to obtain sol.
Example A.42: 0.5 gr of CMC high molecular weight, 0.75 PVA, 0.25 gr Carbopol 934 and 0.2 g poloxamer 407 were added to 88.85 gr of water and mechanically (severe mixing) mixed at 40° C. for up to 1 h to obtain a 1.9 wt % of polymer aqueous solution. 4.0 gr of glycerol and 0.2 gr sorbitol are mixed with the polymers solution for 15 min to obtain sol.
Example A.43: 0.5 gr of CMC high molecular weight, 1.5 PVA, 0.25 gr Pemulene TR1 and 0.25 g poloxamer 407 were added to 83.25 gr of water and mechanically (severe mixing) mixed at 40° C. for up to 1 h to obtain a 2.8 wt % of polymer aqueous solution. 4.0 gr of glycerol and 0.25 gr sorbitol are mixed with the polymers solution for 15 min to obtain sol.
Example A.44: 1.0 gr of HPMC (hydroxypropyl methyl cellulose) added to 92.0 gr of water and mechanically (severe mixing) mixed at 40° C. for up to 1 h to obtain a 1.08 wt % of polymer aqueous solution. 4.0 gr of glycerol are mixed with the polymers solution for 15 min to obtain sol.

### Nanodomains Concentrate Preparation:

Several selected formulations of the so called “concentrates” (oily-phase of all the components except water, buffers and, some antioxidants, preservatives) were used in these experiments. The concentrate may include surfactants, e.g. polysorbate 80 (Tween 80) and other co-surfactants and additives including propylene glycol, diethylene glycol monoethyl ether (Transcutol), lechitins, Dimethicone and glycerol. The concentrates were prepared by mixing all components in their desirable concentration at optimized conditions until a transparent solution-like mixture is obtained.

Example B.1: 5 gr Tween 80, 1.5 gr propylene glycol, 1.5 gr transcutol, 0.8 gr lecithin, 0.8 gr glycerol and 0.4 gr dimethicone were mixed together for 4 hours to obtain 10 gram of nanodomain concentrate consisting of 50 wt % Tween 80, 15 wt % of propylene glycol, 15 wt % of Transcutol, 8 wt % of lecithin, 8 wt % of glycerol and 4% of dimethicone.
Example B.2: 4.5 gr Tween 80, 2.0 gr propylene glycol, 1.2 gr transcutol, 0.5 gr lecithin, 1.0 gr glycerol and 0.8 gr dimethicone were mixed together for 4 hours to obtain 10 gram of nanodomain concentrate consisting of 45 wt % Tween 80, 20 wt % of propylene glycol, 12 wt % of Transcutol, 5 wt % of lecithin, 00 wt % of glycerol and 8 wt % of dimethicone.
Example B.3: 2.8 gr Tween 80, 1.0 gr Tween 20, 1.6 gr Cremophor EL, 2.0 gr propylene glycol, 1.5 gr Transcutol, 0.3 gr dimethicone, 0.5 gr glyceol and 0.3 gr lecithin (phospholipids), were mixed together for 4 hours to obtain 10 gram of nanodomain concentrate consisting of 28 wt % Tween 80, 00 wt % Tween 20, 16 wt % Cremophor EL, 20 wt % of propylene glycol, 15 wt % of Transcutol, 3 wt % dimethicone, 5 wt % glycerol and 3 wt % of lecithin.
Example B.4: 2.5 gr Tween 80, 2.3 gr Tween 20, 18 gr propylene glycol, 1.5 gr transcutol, 0.5 gr oleyl alcohol, 0.5 gr glyceol and 0.9 gr Plurol Oleique CC 497, were mixed together for 4 hours to obtain 10 gram of nanodomain concentrate consisting of 25 wt % Tween 80, 23 wt % Tween 20, 18 wt % of propylene glycol, 15 wt % of Transcutol, 5 wt % oleyl alcohol, 5 wt % glycerol and 9 wt % of Plurol Oleique CC 497
Example B.5: 2.5 gr Tween 80, 2.3 gr Tween 20, 15 gr propylene glycol, 1.5 gr transcutol, 0.5 gr oleyl alcohol, 0.5 gr glycerin, 0.5 gr IPA and 0.9 gr Plurol Oleique CC 497, were mixed together for 4 hours to obtain 10 grams of nanodomain concentrate consisting of 25 wt % Tween 80, 23 wt % Tween 20, 13 wt % of propylene glycol, 15 wt % of Transcutol, 5 wt % oleyl alcohol, 5 wt % glycerol, 5 wt % IPA and 9 wt % of Plurol Oleique CC 497
Example B.6: 2.5 gr Tween 80, 2.3 gr Tween 20, 15 gr propylene glycol, 1.5 gr transcutol, 0.5 gr oleyl alcohol, 0.5 gr glycerin, 0.5 gr DMI and 0.9 gr Plurol Oleique CC 497, were mixed together for 4 hours to obtain 10 gram of nanodomain concentrate consisting of 25 wt % Tween 80, 23 wt % Tween 20, 13 wt % of propylene glycol, 15 wt % of Transcutol, 5 wt % oleyl alcohol, 5 wt % glycerol, 5 wt % DMI and 9 wt % of Plurol Oleique CC 497
Example B.7: 0.3 gr lecithin (phospholipids), 0.7 gr Brij CS 20, 2.0 gr Tween 20, 1.7 gr Tween 60, 0.3 gr oleyl alcohol, 0.6 gr benzyl alcohol, 1.4 gr Transcutol, 1.0 gr IPA and 2.0 gr propylene glycol were mixed together for 4 hours to obtain 10 gr of nanodomain concentrate consisting of 3 wt % lecithin, 7 wt % Brij CS 20, 20 wt % Tween 20, 17 wt % Tween 60, 3 wt % oleyl alcohol, 6 wt % benzyl alcohol, 14 wt % Transcutol, 10 wt % IPA and 20 wt % propylene glycol.
Example B.8: 0.3 gr lecithin (phospholipids), 1.6 gr Brij CS 20, 1.05 gr seteareth-21, 1.7 gr Tween 60, 0.3 gr oleyl alcohol, 0.65 gr benzyl alcohol, 1.4 gr transcutol, 1.0 gr IPA and 2.0 gr propylene glycol were mixed together for 4 hours to obtain 10 gram of nanodomains concentrate consisting of 3 wt % lecithin, 16 wt % Brij CS 20, 10.5 wt % seteareth-21, 17 wt % Tween 60, 3 wt % oleyl alcohol, 6.5 wt % benzyl alcohol, 14 wt % transcutol, 10 wt % IPA and 20 wt % propylene glycol.
Example B.9: 0.3 gr lecithin, 1.2 gr Brij CS 20, 1.2 gr Tween 20, 2.0 gr Tween 80, 0.3 gr oleyl alcohol, 0.6 gr benzyl alcohol, 1.4 gr transcutol, 1.0 gr IPA and 2.0 gr propylene glycol were mixed together for 4 hours to obtain 10 gram of nanodomains concentrate consisting of 3 wt % lecithin, 12 wt % Brij CS 20, 12 wt % Tween 20, 20 wt % Tween 80, 3 wt % oleyl alcohol, 6 wt % benzyl alcohol, 14 wt % Transcutol, 10 wt % IPA and 20 wt % propylene glycol.
Example B.10: 0.3 gr lecithin, 1.2 gr Brij CS 20, 1.2 gr Tween 20, 2.0 gr Tween 80, 0.3 gr oleic acid, 0.6 gr benzyl alcohol, 1.4 gr transcutol, 1.0 gr IPA and 2.0 gr propylene glycol were mixed together for 4 hours to obtain 10 gram of nanodomains concentrate consisting of 3 wt % lecithin, 12 wt % Brij CS 20, 12 wt % Tween 20, 20 wt % Tween 80, 3 wt % oleic acid, 6 wt % benzyl alcohol, 14 wt % transcutol, 10 wt % IPA and 20 wt % propylene glycol.
Example B.11: 0.3 gr lecithin, 0.7 gr Brij CS 20, 2.0 gr Tween 20, 1.7 gr Tween 60, 0.3 gr oleic acid, 0.6 gr benzyl alcohol, 1.4 gr Transcutol, 1.0 gr IPA and 2.0 gr propylene glycol were mixed together for 4 hours to obtain 10 gr of nanodomains concentrate consisting of 3 wt % lecithin, 7 wt % Brij CS 20, 20 wt % Tween 20, 17 wt % Tween 60, 3 wt % oleic acid, 6 wt % benzyl alcohol, 14 wt % Transcutol, 10 wt % IPA and 20 wt % propylene glycol.

### Nanodomain-Polymer Mixture Preparation:

For loaded nanodomain-polymer mixture preparation, a calculated amount of API is added to the “concentrates” and mixed until fully dissolved. The loaded nanodomains were added to a pre-weighted diluted polymer aqueous solution (sol) according to desirable ratios and mixed at optimized conditions (time, speed and temperature) to obtain a homogenous mixtures of polymer solution with nanodomains.

Example C.1: 1.2 gr of Na-DCF was added to 8.8 gr nanodomains concentrate and mixed for 3 hours. The loaded nanodomains was added to 90 gr (8 wt % polymer solution) and mixed at 40° C. for 30 minutes to obtain 100 gr of homogenous mixture (the “sol”).
Example C.2: 0.64 gr of Na-DCF was added to 9.36 gr nanodomain concentrate and mixed for 3 hours. The loaded nanodomains was added to 90 gr 6 wt % polymer solution and mixed at 40° C. for 30 minutes to obtain 100 gr of homogenous mixture.
Example C.3: 0.5 gr of hyaluronic acid was added to 9.5 gr nanodomain concentrate and mixed for 3 hours. The loaded nanodomains was added to 90 gr 5 wt % polymer solution and mixed at 40° C. for 30 minutes to obtain 100 gr of homogenous mixture.
Example C.4: 0.3 gr of lidocaine acid was added to 9.7 gr nanodomain concentrate and mixed for 3 hours. The loaded nanodomains was added to 90 gr 7.5 wt % polymer solution and mixed at 40° C. for 30 minutes to obtain 100 gr of homogenous mixture.
Example C.5: 0.75 gr of terbinafine HCl was added to 6.75 gr nanodomain concentrate and mixed for 1 hours at 40° C. The loaded nanodomains was added to 92.5 gr (5.1 wt % polymer solution) and mixed at 40° C. for 30 minutes to obtain 100 gr of homogenous mixture (the “sol”).
Example C.6: 0.5 gr of terbinafine HCl was added to 4.5 gr nanodomain concentrate and mixed for 1 hours at 40° C. The loaded nanodomains was added to 95 gr (7.1 wt % polymer solution) and mixed at 40° C. for 30 minutes to obtain 100 gr of homogenous mixture (the “sol”).
Example C.7: 0.375 gr of terbinafine HCl was added to 7.125 gr nanodomain concentrate and mixed for 1 hours at 40° C. The loaded nanodomains was added to 92.5 gr (6.5 wt % polymer solution) and mixed at 40° C. for 30 minutes to obtain 100 gr of homogenous mixture (the “sol”).
Example C.8: 0.375 gr of terbinafine HCl was added to 7.125 gr nanodomain concentrate and mixed for 1 hours at 40° C. The loaded nanodomains was added to 92.5 gr (7.0 wt % polymer solution) and mixed at 40° C. for 30 minutes to obtain 100 gr of homogenous mixture (the “sol”).
Example C.9: 0.25 gr of terbinafine HCl was added to 4.75 gr nanodomain concentrate and mixed for 1 hours at 40° C. The loaded nanodomains was added to 95.0 gr (4.2 wt % polymer solution) and mixed at 40° C. for 30 minutes to obtain 100 gr of homogenous mixture (the “sol”).
Example C.10: 0.25 gr of terbinafine HCl was added to 4.75 gr nanodomain concentrate and mixed for 1 hours at 40° C. The loaded nanodomains was added to 95.0 gr (3.7 wt % polymer solution) and mixed at 40° C. for 30 minutes to obtain 100 gr of homogenous mixture (the “sol”).
Example C.11: 0.25 gr of terbinafine HCl was added to 4.75 gr nanodomain concentrate and mixed for 1 hours at 40° C. The loaded nanodomains was added to 95.0 gr (5.3 wt % polymer solution) and mixed at 40° C. for 30 minutes to obtain 100 gr of homogenous mixture (the “sol”).
Example C.12: 0.25 gr of terbinafine HCl was added to 4.75 gr nanodomain concentrate and mixed for 1 hours at 40° C. The loaded nanodomains was added to 95.0 gr (6.3 wt % polymer solution) and mixed at 40° C. for 30 minutes to obtain 100 gr of homogenous mixture (the “sol”).
Examples C.13 to C.15 demonstrate preparation of nutraceutical films, cosmeceutical film and cosmetic films.
Example C.13: 0.4 gr of astaxanthin was added to 3.6 gr nanodomain concentrate and mixed for 1 hours at 40° C. The loaded nanodomains was added to 96.0 gr (1.8 wt % polymer solution) and mixed at 40° C. for 30 minutes to obtain 100 gr of homogenous mixture (the “sol”).
Example C.14: 0.024 gr of curcumin was added to 3.976 gr nanodomain concentrate and mixed for 1 hours at 40° C. The loaded nanodomains was added to 96.0 gr (1.8 wt % polymer solution) and mixed at 40° C. for 30 minutes to obtain 100 gr of homogenous mixture (the “sol”).
Example C.15: 0.024 gr of piperine was added to 3.976 gr nanodomain concentrate and mixed for 1 hours at 40° C. The loaded nanodomains was added to 96.0 gr (1.8 wt % polymer solution) and mixed at 40° C. for 30 minutes to obtain 100 gr of homogenous mixture (the “sol”).
Examples C.16 to C.18 illustrate preparation of crosslinked films and/or films including permeation agent.
Example C.16: 0.375 gr of terbinafine HCl was added to 3.375 gr nanodomain concentrate and mixed for 1 hours at 40° C. The loaded nanodomains, 0.2 gr urea and 0.5 gr lactic acid were added to 95.55 gr (2.8 wt % polymer solution) and mixed at 40° C. for 30 minutes to obtain 100 gr of homogenous mixture (the “sol”).
Example C.17: 0.375 gr of terbinafine HCl was added to 3.375 gr nanodomain concentrate and mixed for 1 hours at 40° C. The loaded nanodomains, 0.2 gr urea, 0.5 gr lactic acid and 0.05 gr citric acid (as crosslinker) were added to 95.50 gr (2.8 wt % polymer solution) and mixed at 40° C. for 30 minutes to obtain 100 gr of homogenous mixture (the “sol”).
Example C.18: 0.375 gr of terbinafine HCl was added to 3.375 gr nanodomain concentrate and mixed for 1 hours at 40° C. The loaded nanodomains, 0.2 gr urea, 0.5 gr lactic acid and 0.1 gr EDTA (as crosslinker) were added to 95.45 gr (2.8 wt % polymer solution) and mixed at 40° C. for 30 minutes to obtain 100 gr of homogenous mixture (the “sol”).

### Film Casting and Drying Procedure:

For film preparation, a calculated amount of nanodomain-polymer mixture was casted on a cleaned and treated casting surface (silicon, glass, Teflon and others) and dried at optimized conditions (temperature, time and controlled conditions for fast aqueous phase evaporation). The film forming process occurs during evaporation of the diluent (water and part of the volatile solvents in this case). Drying conditions, such as time, temperature and casting surface coating were optimized for each specific film composition.

For example: 40 gr of gelatin-nanodomains mixture was casted on a Teflon mold and dried at RT for 48 hours.

Table 1 below summarizes the formulations tested.

TABLE 1
Tested formulation properties

| | Film | | | Permeating | | API in |
|---|---|---|---|---|---|---|
| | formers | Plasticizers | Formulation | agents | Crosslinkers | Film |
| Film | [wt %] | [wt %] | [wt %] | [wt %] | [wt %] | [wt %] |
| Native Film | 100 | — | — | — | — | — |
| Native Film + | 5-15 | 85-95 | — | — | — | — |
| plasticizer | | | | | | |
| Native Film + | 25-35 | 45-60 | — | 6-10 | 0-2 | — |
| plasticizer + | | | | | | |
| permeating | | | | | | |
| agents + | | | | | | |
| Crosslinkers | | | | | | |
| Film embedded | 10-25 | 40-55 | 10-30 | — | — | — |
| with empty | | | | | | |
| nanodomains | | | | | | |
| (no permeating | | | | | | |
| agents or | | | | | | |
| crosslinkers) | | | | | | |
| Film embedded | 15-25 | 30-45 | 26-33 | 2-8 | 0-2 | |
| with empty | | | | | | |
| nanodomains | | | | | | |
| (with | | | | | | |
| permeating | | | | | | |
| agents or | | | | | | |
| crosslinkers) | | | | | | |
| Film | 10-25 | 40-55 | 10-30 | | | 0.1-12 |
| embedded | | | | | | |
| with | | | | | | |
| nanodomains | | | | | | |
| with API | | | | | | |
| Film embedded | 15-25 | 30-45 | 26-33 | 2-8 | 0-2 | 0.1-3.5 |
| with | | | | | | |
| nanodomains | | | | | | |
| with API (with | | | | | | |
| permeating | | | | | | |
| agents or | | | | | | |
| crosslinkers) | | | | | | |

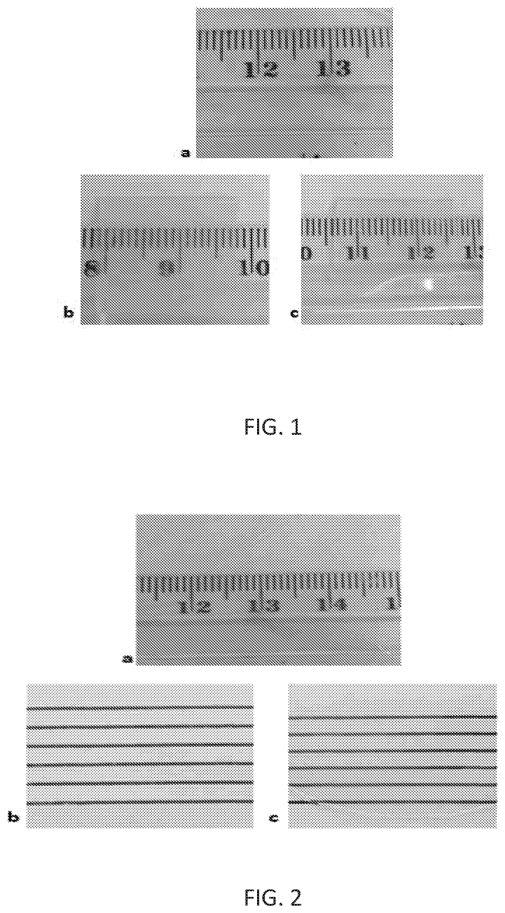

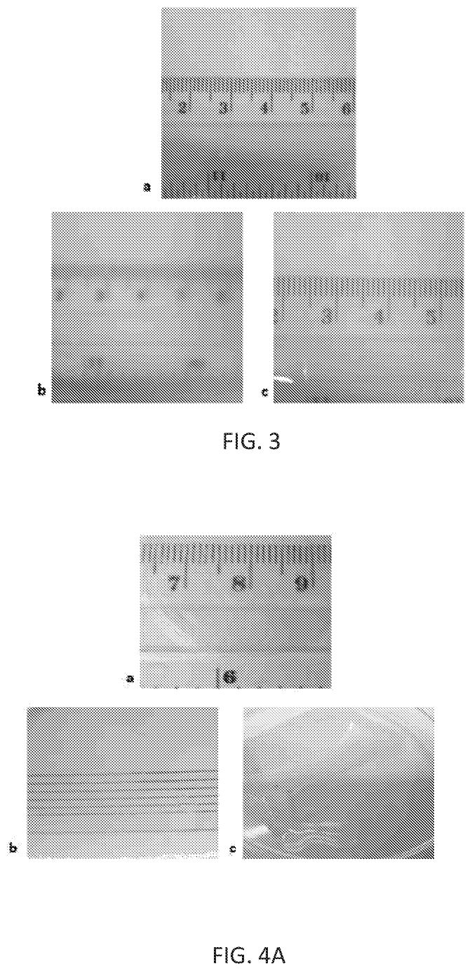

FIG. 1-FIG. 4 show photographs demonstrating the transparency of the casted polymer films. As seen from FIG. 1 and FIG. 2, when gelatin is used as the film-forming polymer, the transparency of the film remains unchanged when embedded with empty as well as loaded nanodomains. This indicates that the nanodomains are homogeneously dispersed within the film and that their structural integrity is maintained within the film. As seen from FIG. 3, when sorbitol was used as a plasticizer, the film embedded with empty nanodomains was slightly turbid. However, once loaded with Na-DCF, which is an amphiphilic ‘structure builder’ molecule assisting the formation of more “ordered” nanodomains, the film became transparent and homogeneous (see b and c). Similar results were obtained for film loaded with permeating enhancers such as DMI and Transcutol.

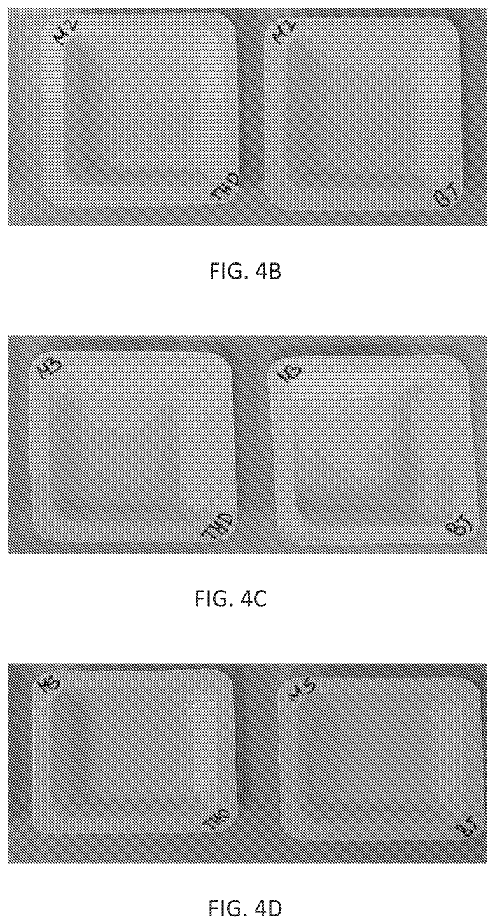

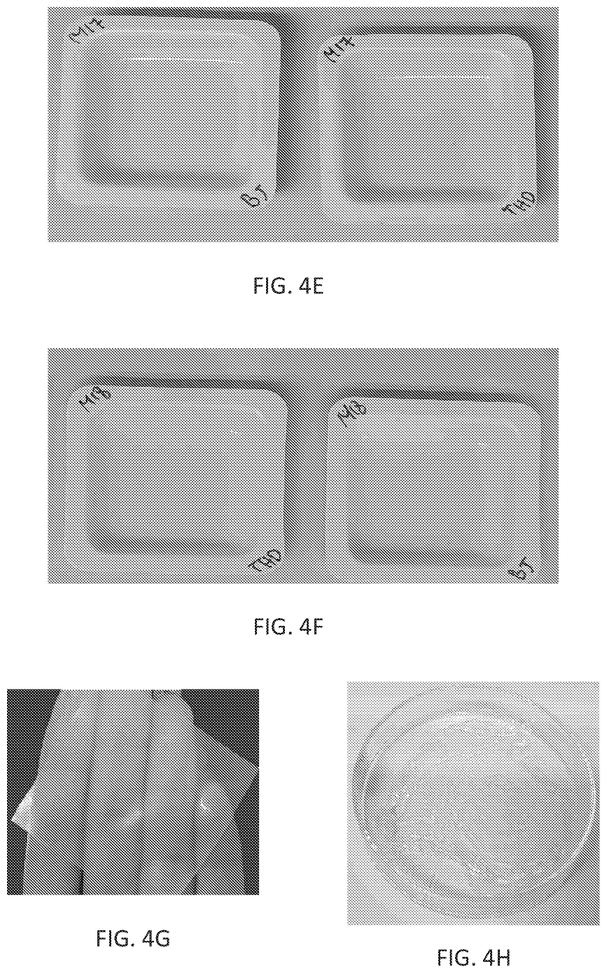

However, as seen from FIG. 4A, when PVA is used as the film-forming polymer, the film becomes turbid as a result of nanodomains embedment (API-loaded or not), thus indicating that PVA causes the nanodomain to be non-homogeneously dispersed within and/or over the film, and/or to compromise the structural integrity of the nanodomains.

However, as seen from FIG. 4B, when a combination of polymers including gelatin, PVA and poloxamer 407 was utilized and glycerol as plasticizer (example C.8) was added, a smooth and transparent film was obtained, whether the nanodomains concentrate was prepared using THD (example B.4) (left panel) or BJ (example B.11) right panel, in each case loaded with 5 wt % terbinafine HCl (see example C8).

Similarly, as seen from FIG. 4C, when a combination of polymers including CMC and PVA was used and a plasticizer (glycerol) added, clear and transparent film was obtained, whether the nanodomains concentrates was prepared using THD (left panel) or BJ (right panel), each loaded with 5 wt % terbinafine HCl (see example C.9).

Clear and transparent film was also obtained when a combination of polymers including CMC and HEC was utilized and glycerol as plasticizer added, whether the nanodomains concentrates was prepared using THD (left panel) or BJ (right panel), in each case loaded with 5 wt % terbinafine HCl (example C.9), as seen from FIG. 4D.

Similarly, clear and transparent film was also obtained, when HEC was used as the polymer, alone (FIG. 4E) or in combination with PVP (FIG. 4F), using glycerol as a plasticizer and tested using either THD nanodomain concentrates (left panel) or BJ nanodomain concentrates (right panel), in each case loaded with 5 wt % terbinafine HCl.

FIG. 4G shows the film obtained using a crosslinked using citric acid polymers, here CMC, PVA, poloxamer 407 and TR1 (see example C.17), wherein glycerol and sorbitol were added as plasticizers, urea and lactic as penetrating enhancers. The nanodomains concentrate is based on BJ-h system (example B.11).

As seen from FIG. 4H, the film forming capabilities of the herein disclosed polymers are unique as certain polymers, such as hydroxypropyl methylcellulose (HPMC) are unsuitable as film formers and resulted in lumpy unsatisfactory film.

### Example 2: Mechanical Properties of Gelatin-Nanodomain Film

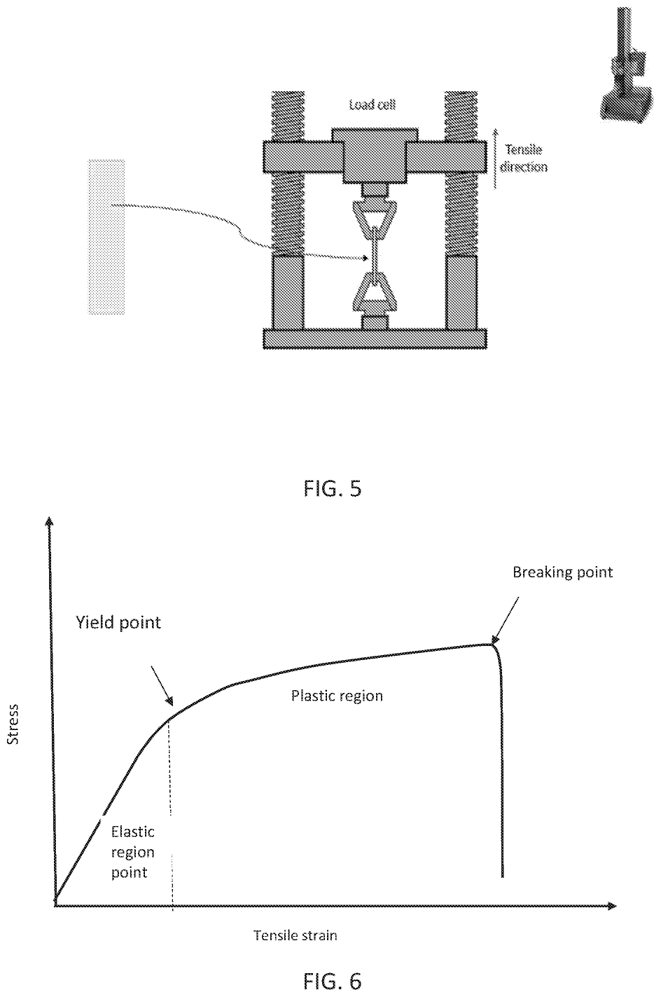

The goal of this study was to determine the effect of nanodomains embedment on the mechanical properties of film. Instron instrumentation was used for testing. During the test, a pulling force was applied to the film and the film's strength, and elasticity (stress) was measured, essentially according to the setup depicted in FIG. 5. An illustrative stress-strain curve is depicted in FIG. 6. As seen from the curve, the first stage is the elastic region during which the stress is proportional to the strain; that is, the material undergoes only elastic deformation. In this region, the stress mainly increases as material elongates. As the strain accumulates, the stress reaches the tensile strength, upon which the breaking point is reached and the material fractures. After fracture, percent elongation and reduction in section area can be calculated.

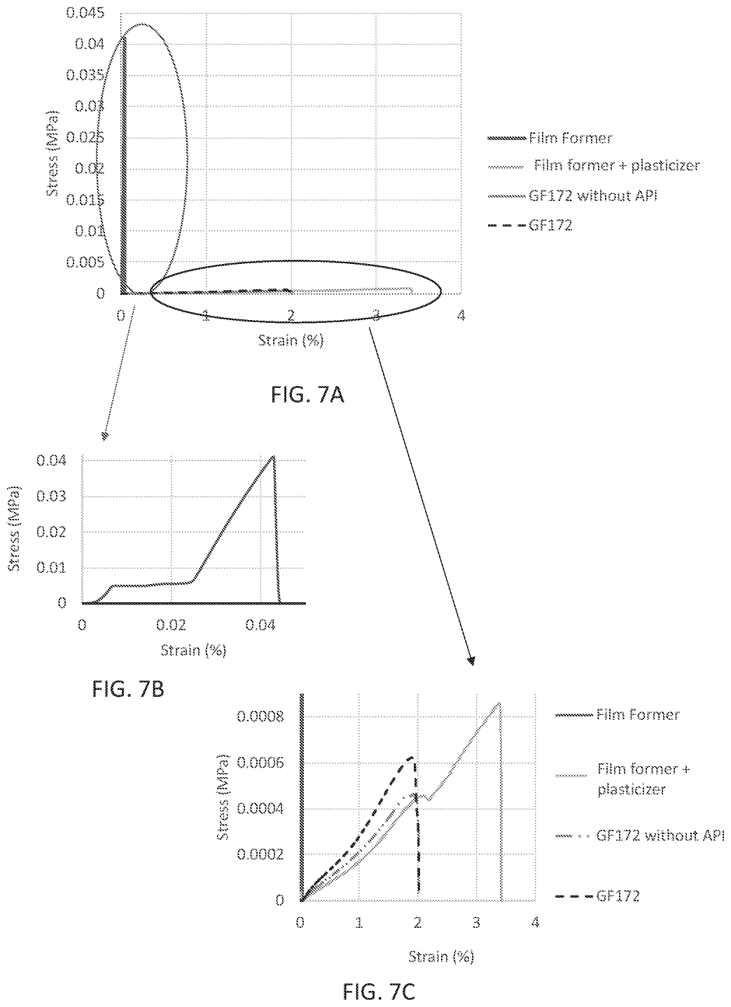

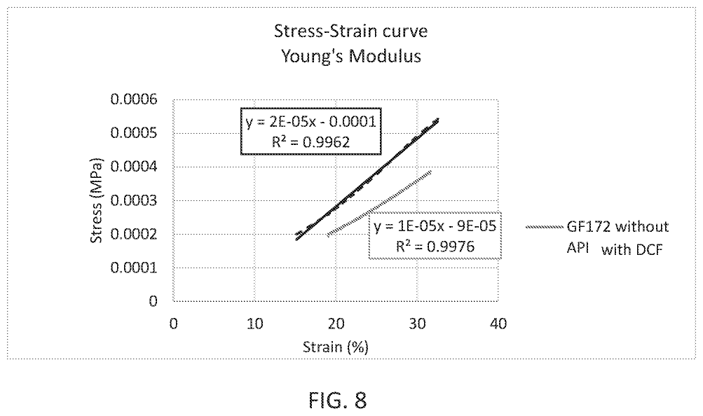

As seen from the graphs of FIG. 7A-FIG. 7C, the nanodomain embedded films have an elasticity similar to that of the native films formed with plasticizer, thus indicating that the embedment of nanodomains, albeit weakening the film, increases its elasticity. Similarly, as seen from FIG. 8, the Young's Modulus of elasticity nanodomains is even further increased when the nanodomains are loaded with DCF. The mechanical properties of the different films are summarized in Table 2 below.

TABLE 2
mechanical properties of films

| | | Stress at | Modulus |
|---|---|---|---|
| | Maximum | breaking | young, |
| | elongation | point, σ_{B} | E |
| Film | EL[%] | [MPa] | [MPa] |
| Native Film | 0.043 | 0.0411 | 2.3294 |
| Native Film + | 3.394 | 0.0009 | 0.0004 |
| plasticizer | | | |
| Film embedded with | 1.924 | 0.0004 | 0.0003 |
| empty nanodomains | | | |
| Film embedded with | 1.912 | 0.0006 | 0.0004 |
| nanodomains with | | | |
| Na-DCF | | | |
| Native Film + | 98.89 | 0.74 | 0.58 |
| plasticizer + | | | |
| permeating agents + | | | |
| Crosslinkers | | | |
| Film embedded | 175.74 | 0.64 | 0.37 |
| with empty | | | |
| nanodomains (with | | | |
| permeating | | | |
| agents or crosslinkers) | | | |
| Film embedded with | 77.09 | 0.23 | 0.17 |
| nanodomains with | | | |
| API (with | | | |
| permeating agents and | | | |
| plasticizers), not | | | |
| crosslinked | | | |
| Film embedded with | 82.28 | 0.37 | 0.36 |
| nanodomains with | | | |
| API (with | | | |
| permeating agents and | | | |
| plasticizers), crosslinked | | | |

### Example 3: Structural Characterization of Nanodomains

SAXS is a small-angle scattering technique by which nanoscale density differences in a structure can be quantified. This means that it can determine nanoparticle size distributions, resolve the size and shape of (monodisperse) macromolecules, determine pore sizes, characteristic distances of partially ordered materials, etc. This is achieved by analyzing the elastic scattering behavior of X-rays when travelling through the material, recording their scattering at small angles (typically 0.1-10°). Depending on the angular range in which a clear scattering signal can be recorded, SAXS is capable of delivering structural information of dimensions between 1 and 100 nm.

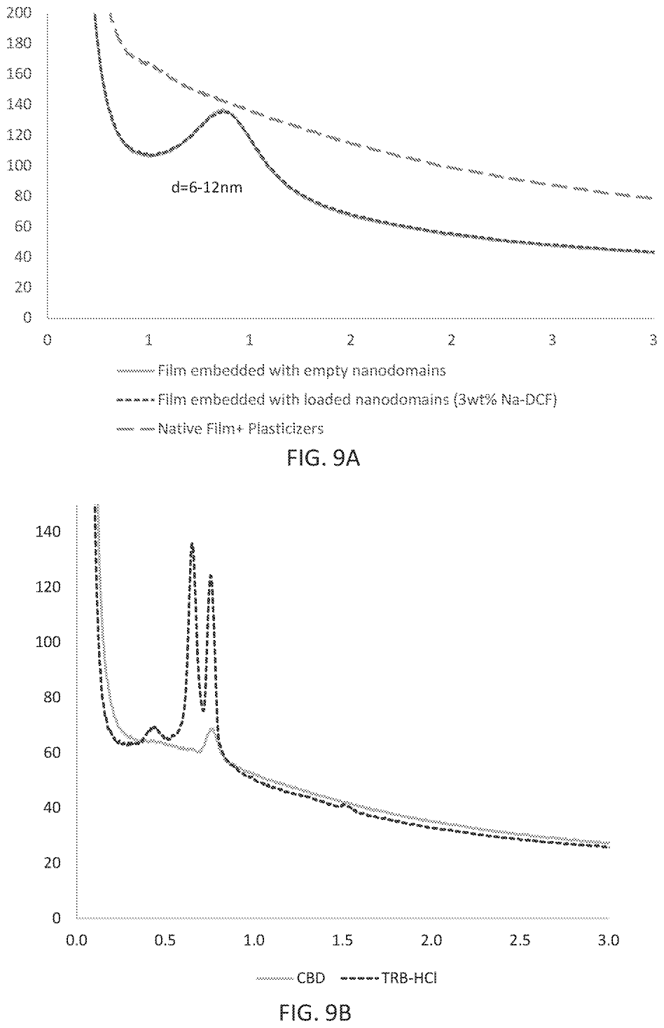

As seen from FIG. 9, the density differences of the film change upon embedment with nanodomains (whether loaded or empty), thereby clearly indicating the that nanodomain structure is maintained within the film. That is, the peaks (signals) obtained for films embedded with nano-domains (empty as well as loaded) indicate the presence of nano-structures within the film. However, in case of native film (no nanodomains embedded) there is no signal suggesting that absence of structure.

SD-NMR is an advanced analytical tool recently adopted to determine the mobility (diffusivity) of each of the ingredients in the nanostructure at any given condition (any water dilution, temperature, pH, etc.), to obtain information on the location of the API within the nanodomains prior to being loaded, and after being released (discharged) from the film.

Very low diffusion coefficients (Dt, diffusivity at a given water dilution or time) in the range of 10⁻¹¹ cm/sec are indicative of an ingredient being “restricted” in its mobility, either because it is located within the core of the nanodomains, or due to interactions with an adjacent ingredient within the nanodomain. While high diffusion coefficient (Dt) such as in the range of ca 10⁻⁹ cm/sec of the ingredient being free to move and thus of an absence of interactions with other components, diffusivity coefficients in between these values reflect the degree of freedom of the component, or its interaction at the interface. For example, if water is locked in the core of the nanodomains, its diffusion coefficient (Dt) will be closer to 10⁻¹¹ cm/sec while, if water is free in the continuous phase, its Dt will be in the range of 10⁻⁹. The technique allows to determine if the API is free or bound to the surfactant or to the nanodomains interface, before being embedded in the film and after being released/dissoluted from the film.

The SD (diffusivity) of only the three major components composing the nanodomains (water, surfactant, API) was measured and calculated for: a) the empty nanodomains reconstituted after film dissolution—blue bars and, b) nanodomains loaded with Na-DCF reconstituted after film dissolution—orange bars.

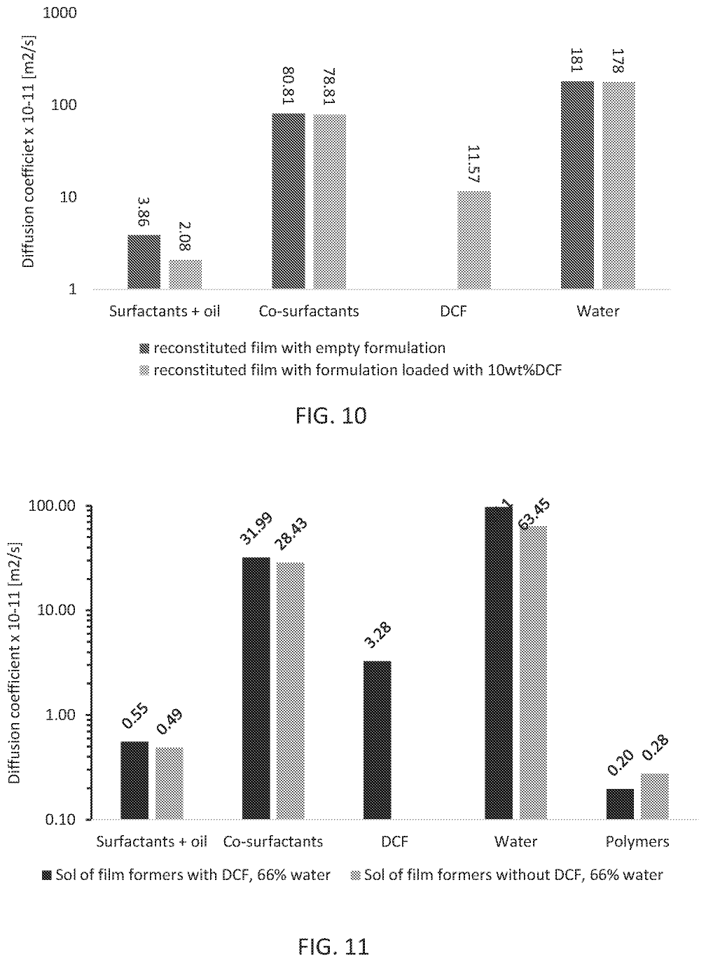

As seen in FIG. 10:

1) The diffusivity coefficient of the API (Na-DCF) is in between that of water and of the surfactant, yet closer to that of the surfactant, thus indicating that the API is positioned in the outer layer of the nanodomain interface (close to the headgroup) in vicinity to the surfactants. That is, since the DC of the API is closer to that of the surfactant of the nanodomains (10\hat{ }(−11)), than to the DC of water (10\hat{ }(−9)), it indicates that the API is closer to the head group, but yet in the interface of the nanodomains.

2) loading of the nanodomains with Na-DCF causes a reduction in the diffusivity coefficients thus indicating that the surfactants/API binding and the API is now located deeper at the interface.

3) water, in both reconstituted mixtures, is relatively free.

4) Upon dilution of the nanodomains the co-surfactant tends to “migrate” out of the nanodomains. This ability is an important trait in that it enhances API release during skin contact (the API having DC of 10⁻¹⁰).

As seen from FIG. 11, showing the diffusivity coefficients of pre-casting mixtures (sols), at a water content of 66 wt %, there are no significant differences between the diffusion coefficients of the components in the empty and DCF loaded systems. Water is slightly less bound in the DCF loaded nanodomains (97×10⁻¹¹ m²/s versus 63×10⁻¹¹ m²/s), suggesting that the incorporation of DCF at 66 wt % water may lead to faster drying time of the film as compared to the empty embedded nanodomains. In addition, one can see that the diffusivity coefficients of surfactant and oil is very low, indicating them being strongly bound to the nanodomains. The diffusivity coefficients of the DCF is lower than that of the co-surfactant but slightly higher than that of the surfactants+oil, suggesting that DCF may be bound to an outer part of the surfactant head group—namely the “palisades”.

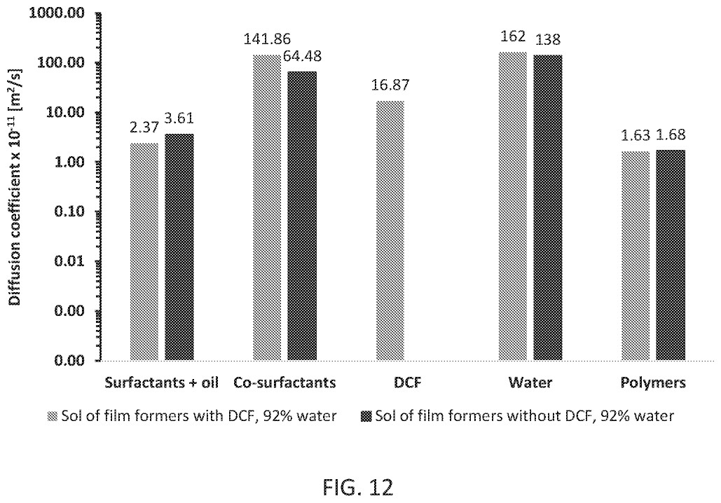

As seen from FIG. 12, showing the diffusivity coefficients of pre-casting mixtures (sols), at 92 wt % water, the differences in diffusion coefficients of the nanodomain components is slightly more pronounced when comparing empty nanodomains to DCF-loaded nanodomains. In the presence of DCF, the mobility of surfactants molecules is slightly lower as compared to the empty nanodomains. The diffusivity of the co-surfactants in the loaded nanodomains is far greater (diffusion coefficients 2.2 times higher than that of the empty nanodomains). The diffusion coefficients of DCF itself is also slightly higher, indicating that at high water contents the DCF is much more mobile, yet, still located at the interface of the nanodomains.

That is, when comparing the pre-casting mixtures at 66 wt % water dilution to that of 92 wt % water dilution, it is noticeable that the mobility of all components, including water is higher. Without being bound by any theory, this difference is due to the form of the nanodomains that assume a spherical oil/water structure at 92 wt % water dilution compared to dense, non-spherical nano-structures at 66 wt % water dilution.

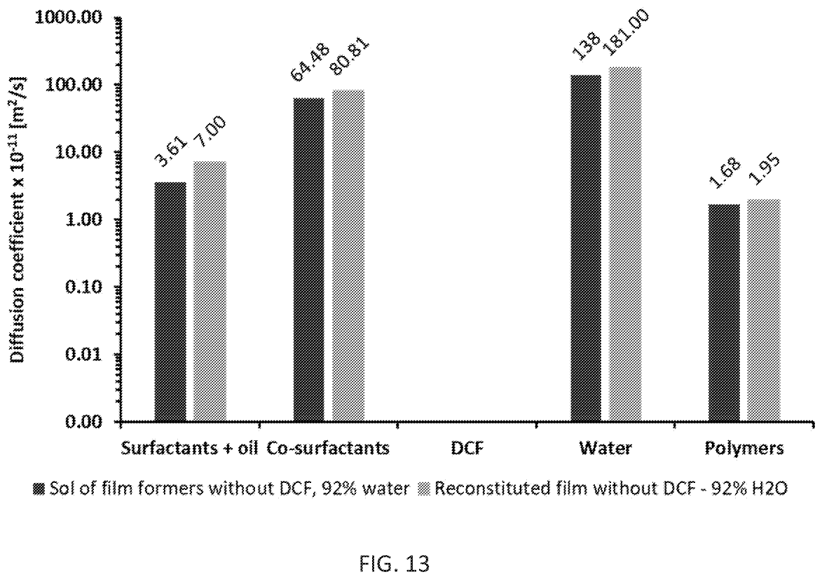

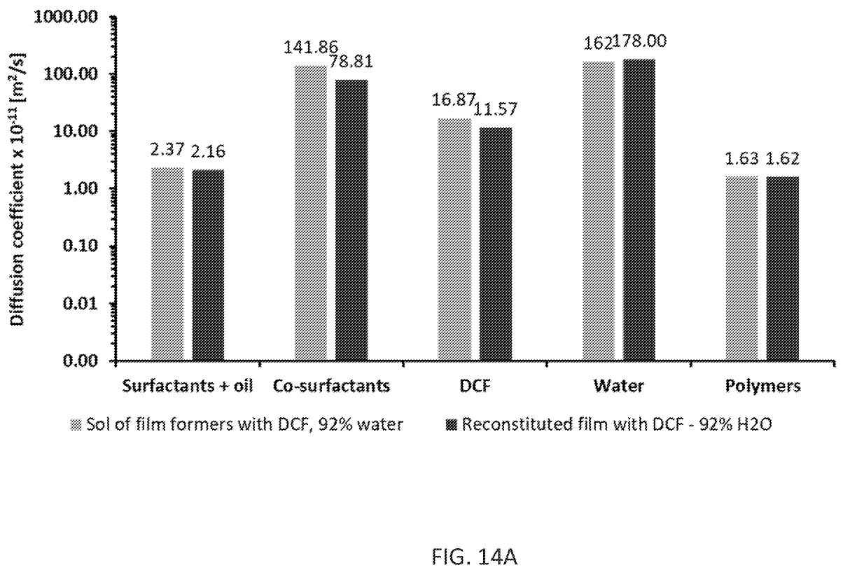

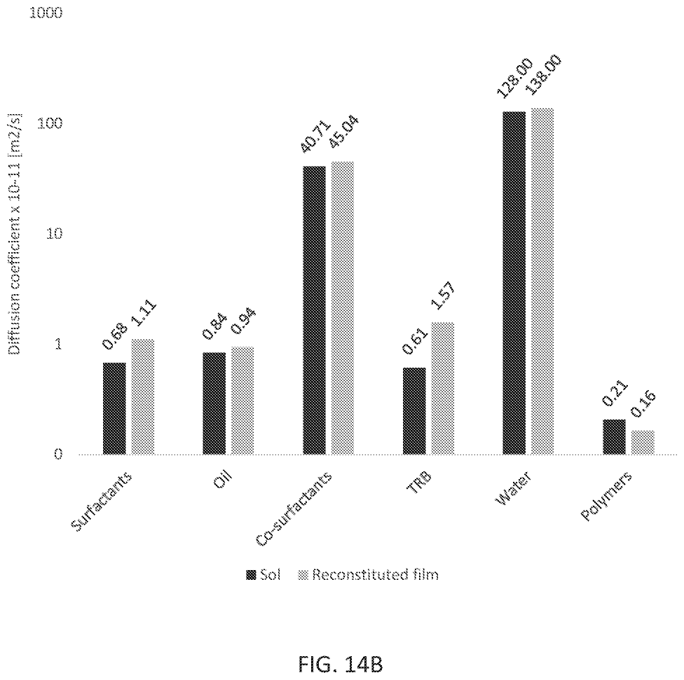

As seen in FIG. 13, showing the diffusivity coefficients of pre-casting mixtures (sols) and of mixtures obtained after film dissolution, in the absence of DCF, reconstitution of the film (film dissolution) leads to higher mobility of all the components in the system, including the water, as compared to that of the pre-casting mixture.

As seen in FIG. 14, when comparing the diffusivity coefficients in a pre-casting mixture containing DCF-loaded nanodomains to the diffusivity coefficients in a mixture of reconstituted DCF loaded nanodomains (after film dissolution), no pronounced difference was observed. The only difference was the diffusivity coefficients of the co-surfactants, the mobility of which was higher in the pre-casting mixture as compared to the mixture obtained after film dissolution. This may be due to the rearrangement of the polymeric matrix or to ci-surfactant-polymer interactions.

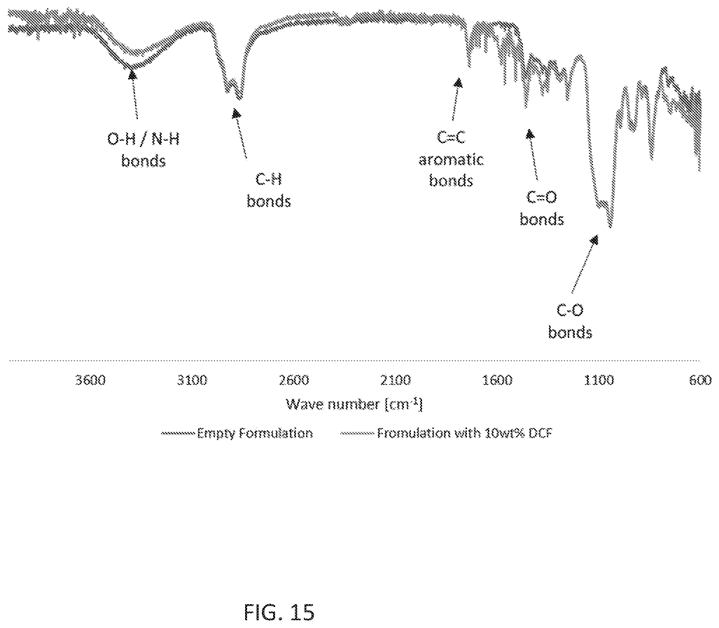

FTIR spectra represent the vibrational movements of certain bonds within a molecule (for example C-H stretching) as a result of its embedment in the nanodomains and thus enables one to determine whether the film imparts interactions with the surfactants or the API. Inter-molecular interactions between components in the nanodomains results in changes in the vibrational spectra. As seen from FIG. 15, showing FITR spectra of nanodomain concentrates, there is essentially no influence of Na-DCF on the OH and NH vibrational energy in the nanodomains concentrate (3200 cm-¹ vs 3300 cm-¹). In fact, even the hydrogen bonds are essentially unaffected by Na-DCF-loading. Moreover, the absolute values of the vibrations are relatively low (3300 cm⁻¹ and 3180 cm⁻¹ respectively).

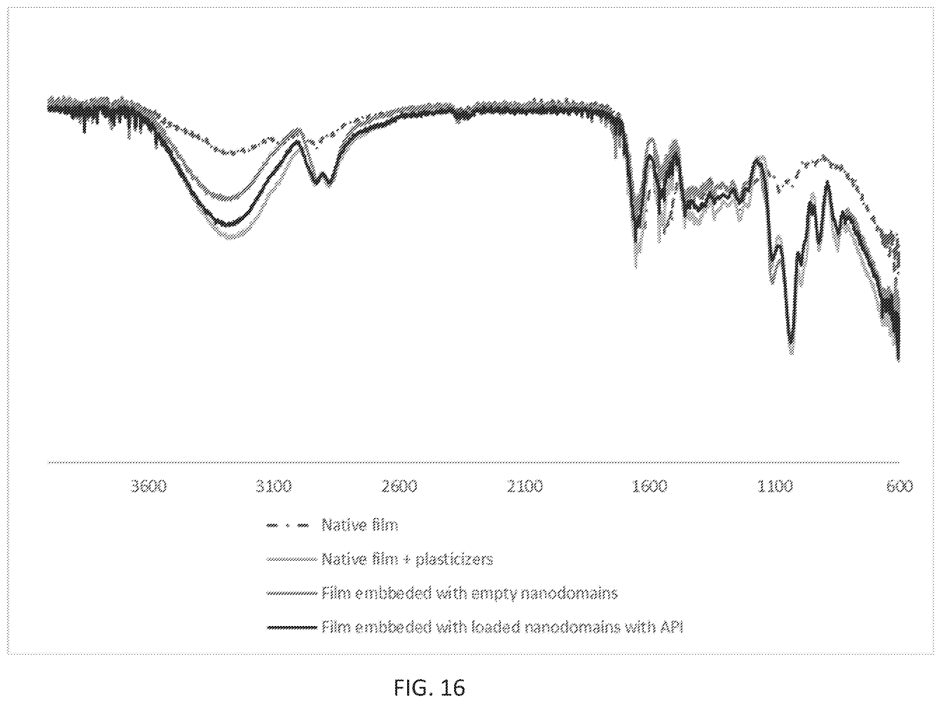

Furthermore, as seen from FIG. 16, showing FTIR spectra of films, native or embedded with nanodomains, the embedment of nanodomains, whether empty (film no DCF—grey line) or loaded (Film with DCF yellow line), intensifies and broadens the signal vis-à-vis the native film (blue line) in a similar manner to the signal obtains for the film with plasticizer (film+plasticizer—orange line (about 3150 cm⁻¹). These results indicate that the nanodomains are incorporated into the film without causing chemical changes of the film.

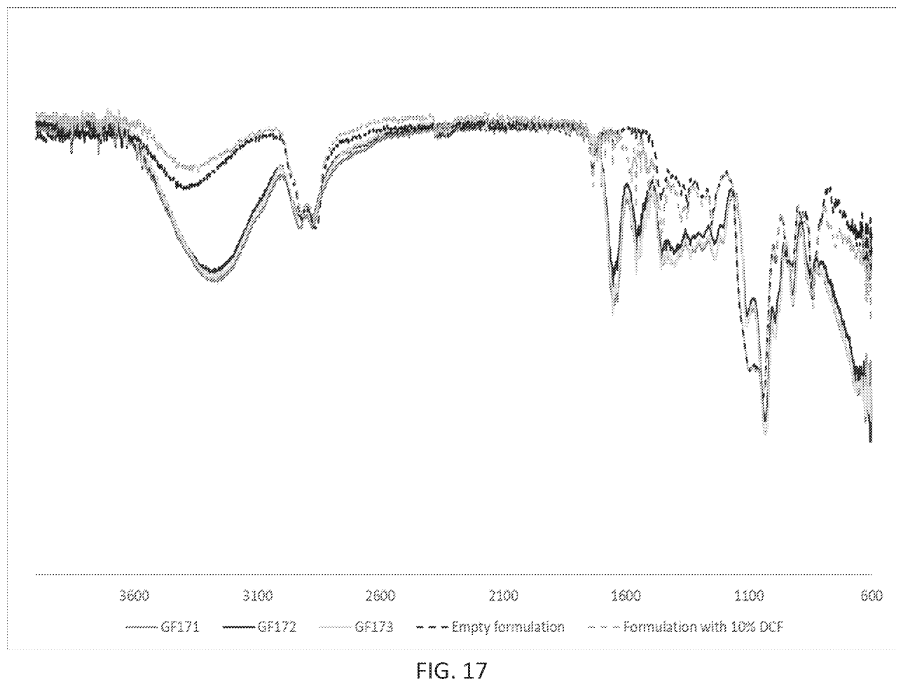

FIG. 17, shows FTIR spectra obtained when measuring films made with different co-film formers. As seen, the peaks obtained when measuring spectra of films (regardless of which film former was utilized (GF171-gelatin and PVP-K30 as co-film former; GF172 PVP and poloxamer 188 as co-film former and GF173-PVP and poloxamer 407 as co-film former) are essentially unchanged.

However, more intense peaks are obtained when measuring spectra of nanodomain concentrates (Empty formulation (yellow line) and Formulation with 10 wt % DCF (orange line) as compared to the film formulation; and the vibrations are slightly shifted (3400 cm⁻¹ for concentrates 3200 cm⁻¹ for films). This is particularly evident for the O—H and N—H bonds, as well as for the carbonyl and amides transmission ranges. This indicated that the presence of film generates stronger molecular interactions than that obtained for nanodomain concentrates.

### Example 4: Ex-Vivo Permeation Study

The goal of this study was to detect and quantify the release of API from films and the amount of API which penetrated through pig skin. Ex-vivo skin permeation studies were performed according to standard protocol using Franz diffusion cells, which enables the detection of drug concentration in the skin, penetration of the drug, the rate of drug transport across the skin, and permeation. Pig skin dermatome with a thickness of 500-700 mm was used.

For the determination of drug permeation to the receptor cell medium (referred as RC) and the permeation into the skin (referred as skin), the RC medium was collected as well as the skin after all drug residues were removed. RC medium was analyzed without further treatments beside a dilution when required. The skin was soaked in dissolution medium, shaken for a couple of hours and sonicated for 30 min. Finally, the dissolution medium was separated from the skin and filtered through 0.45 μm membrane. The obtained medium was analyzed by HPLC for the determination of drug levels.

The drug delivery efficiency was tested for three different film formulations, forming films with different porosity and compared to that of the commercial product Volatol™. As seen from FIG. 18, showing exemplary microscope images of films embedded with DCF loaded nanodomains a) GF171 (contains PVP only) b) GF172 (contains PVP and poloxamer 188) and c) GF173 (contains PVP and poloxamer 407), formulation GF173 form films with the largest ports as compared to the other two film formulations.

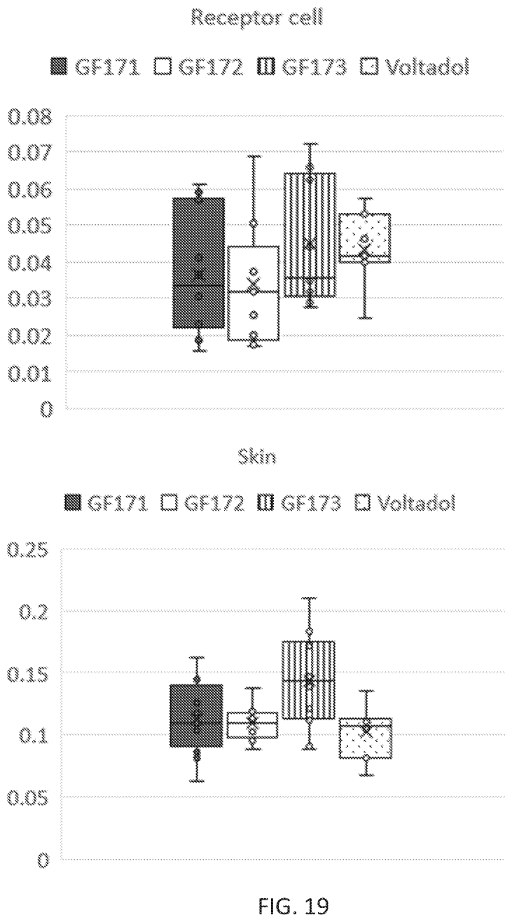

As seen from FIG. 19, superior permeation of DFC was obtained for all of the three herein disclosed nanodomain film formulations, as compared to Volatol™. Moreover, formulation GF173, having films with large pores, provided the best permeation.

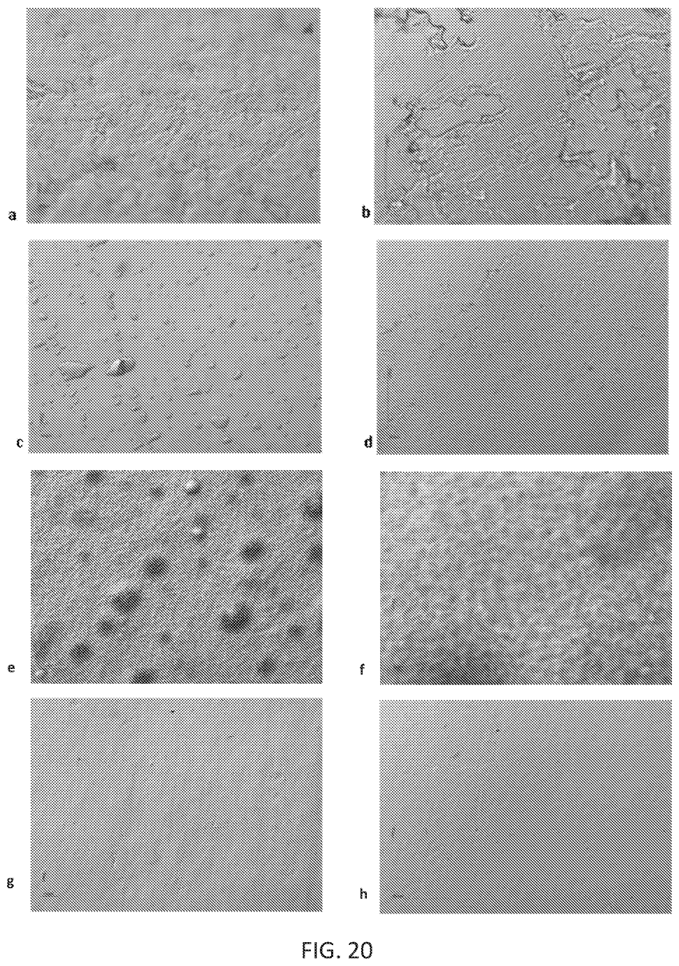

FIG. 20 shows microscope images of: a) native film (magnitude×100), b) native film (magnitude×400, c), native film+plasticizers (magnitude×100), d), native film+plasticizers (magnitude×400), e), film with empty nanodomains (magnitude×100), f), film with empty nanodomains (magnitude×400), g), film with loaded nanodomains with 3 wt % Na-DCF (magnitude×100), h) film with loaded nanodomains with 3 wt % Na-DCF (magnitude×400). As seen from FIG. 20, embedment of the nanodomains (30 wt %) within the film improves homogeneity and uniformity of the film in a similar manner as addition of the plasticizers.

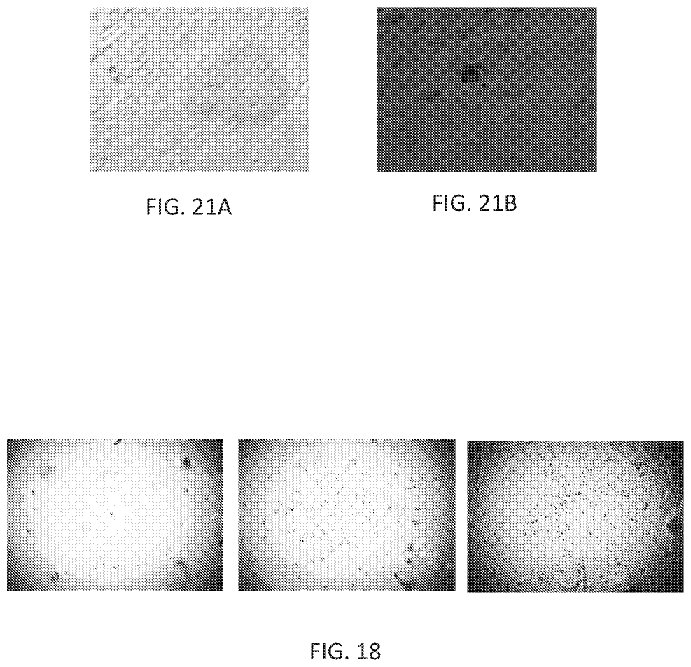

FIG. 21A shows microscope images of a film based on PVA, CMC and poloxamer 407 with loaded nanodomains with 1 wt % terbinafine HCl (magnitude×100).

FIG. 21B shows microscope images of a film based on PVA, CMC and poloxamer 407 with loaded nanodomains with 1-wt % terbinafine HCl (magnitude×400).

As seen FIG. 21A and FIG. 21B, embedment of the nanodomains (30 wt %) within the film results in homogeneous and uniform films.

Drug delivery efficiency was demonstrated for the API terbinafine HCl.

Several film systems were examined in the Franz diffusion model and permeation efficiency compared to that of the commercial gel-product Lamisil Once.

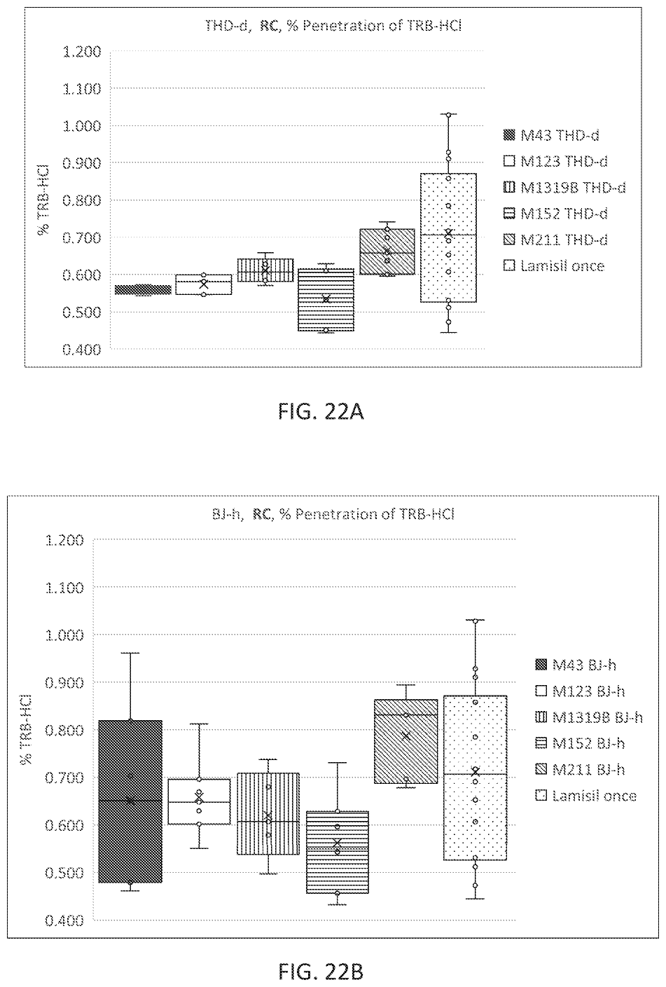

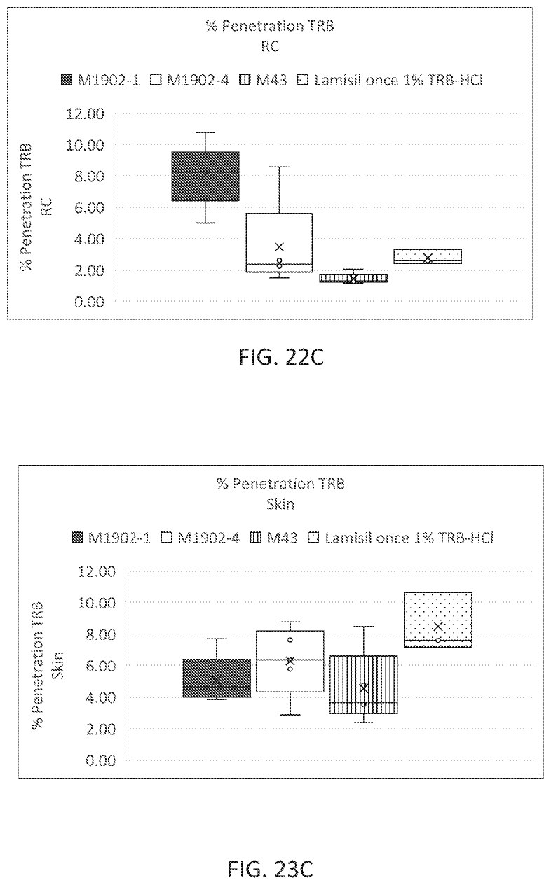

As seen from FIG. 22A, Terbinafine HCl (TRB) permeation (% from applied dose) into receptor cells for THD-d nanodomains system (prepared according to Example B.4) embedded within different polymeric matrices (TRB concentration 1.1 mg/cm²), namely: CMC, PVA and poloxamer 407 based films (M43—dark gray bars), CMC, PVP K30 and poloxamer 407 based films (M123—white bars), CMC, HEC, PVP K30 and poloxamer 407 based films (M1319B—bars with vertical stripes), CMC, PVP K30 and Pemulen TR2 based films (M152—bars with horizontal stripes), or films based on gelatin, PVP K30 and poloxamer 407 (M211—spotted bars), as compared to Lamisil Once product (green).

The receptor cell mimics the blood stream, meaning that permeation into the receptor cell medium is indicative of systemic permeation of the drug. Here it is seen that all the film systems permeate lower levels of TRB-HCl as compared to the control, suggesting that this system limits the systemic exposure of the TRB-HCl. It is noted that a slow release profile of the bioactive may be desirable for delivery of API's requiring a slow and persistent release profile, such as, for example, analgesics for treatment of chronic pain.

As seen in FIG. 22B, similar results were obtained using a BJ-h nanodomains system except for the M211 (bars with diagonal stripes) system that showed higher levels of TRB-HCl penetration compared to the control indicating that the M211 systemic may be suitable for drugs requiring a faster release profile (such as analgesics for immediate relief) while other film compositions may be advantageous for APIs requiring a slower release profile.

As seen in FIG. 22C, permeation into the receptor cells was significantly increased in films to which lactic acid and urea were added as permeation enhancers namely: PVA, CMC, poloxamer 407 and TR1 based film embedded with BJ-h nanodomains system (TRB concentration was 2.7 wt % (M1902-1—dark gray bars), PVA, CMC, poloxamer 407 and TR1 based films embedded with BJ-h nanodomains system (TRB concentration was 3 wt % (M1902-4—white bars), as compared to a same film without permeation agent (M43 bars with vertical stripes) and Lamisil Once (control—dotted bars). Without being bound by any theory, it may be suggested that the permeating enhancers either help penetrating the skin membrane or improve the binding of the system to components in the membrane, and by that, facilitates the greater permeation of TRB-HCl. It is noted that films containing permeating enhancers may be particularly favorable for API requiring transdermal delivery and/or for APIs requiring rapid release.

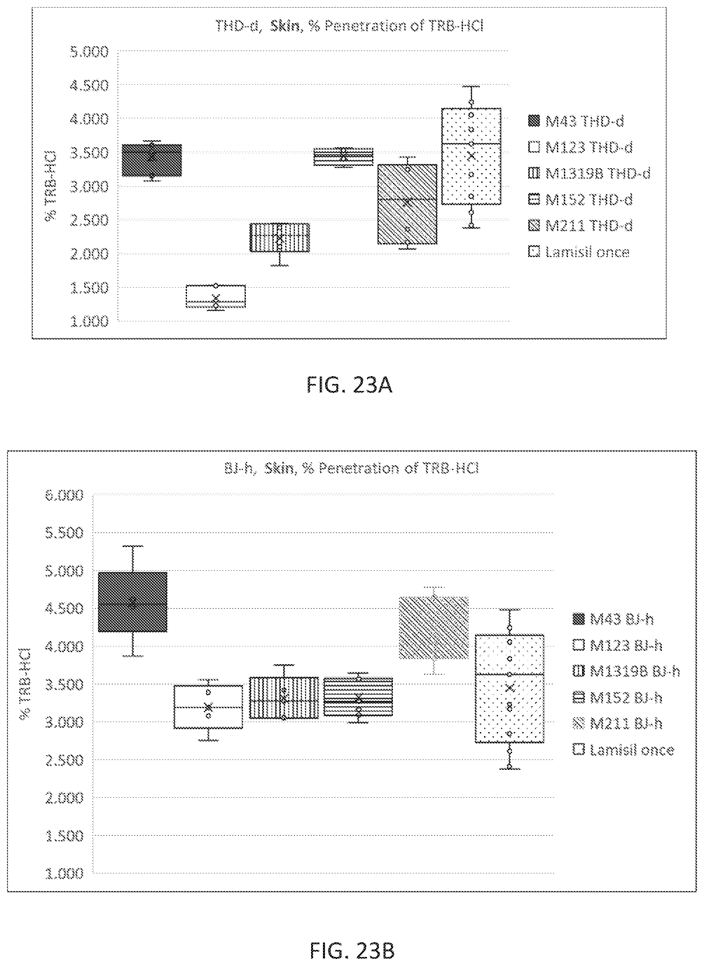

Permeation into skin was also tested. FIG. 23A depicts TRB permeation (% from applied dose) into skin for THD-d nanodomains system embedded within different polymeric matrices (TRB concentration was 1.1 mg/cm²), namely: CMC, PVA and poloxamer 407 based films (M43—dark gray bars), CMC, PVP K30 and poloxamer 407 based films (M123—white bars), CMC, HEC, PVP K30 and poloxamer 407 based films (M1319B—bars with vertical stripes), CMC, PVP K30 and Pemulen TR2 based films (M152—bars with horizontal stripes). As seen from FIG. 23A, a similar penetration of TRB-HCl into the skin as that of the control, was observed for two film systems (M43 and M152) (dotted bars), while the other three systems showed lower TRB-HCl penetration. That is, different polymer matrices provide different levels of TRB-HCl permeation. This advantageously indicates that the films can be specifically designed to provide a desired release profile (e.g. transdermal/topical and slow/fast release) and may thus be customized based on API-demands.

As seen in FIG. 23B, all the systems embedded with BJ-h nanodomains showed similar penetration profile to that of or increased penetration (as in the case of M43 (drak gray bars) and M211—bars with diagonal stripes), as compared to the control (white bars).

In general, TRB-HCl penetration was greater for films including BJ-h nanodomains as compared to the films including THD-d nanodomains suggesting that the release of the API from embedded BJ-h nanodomains was less restricted as compared to embedded THD-d nanodomains.

Furthermore, as seen from FIG. 23C, similarly to the receptor cell test, permeation into the skin was significantly increased in films to which lactic acid and urea were added as permeation enhancers), as compared to the permeating enhancers-free films. It is noted that, in the presence of permeation agents/enhancers, penetration into skin is lower than that of the control. This suggests that, in the presence of the permeation agents/enhancers, the nanodomains rapidly cross the skin, and the API is released in the RC. This behavior is required for transdermal applications and is thus particularly suitable for APIs the absorption of which is decreased when administrated orally due to first pass metabolism (e.g. antibiotics).

An additional test which provide information on the release rate of the API is performed according to additional standard protocol using Franz diffusion cells, which enables the detection of drug concentration in the skin, penetration of the drug, the rate of drug transport across the skin, and permeation. Here, the receptor cell medium is sampled every few hours for 24-48 hours. The samples are analyzed, and the API concentration is determined, and penetration profile plotted.

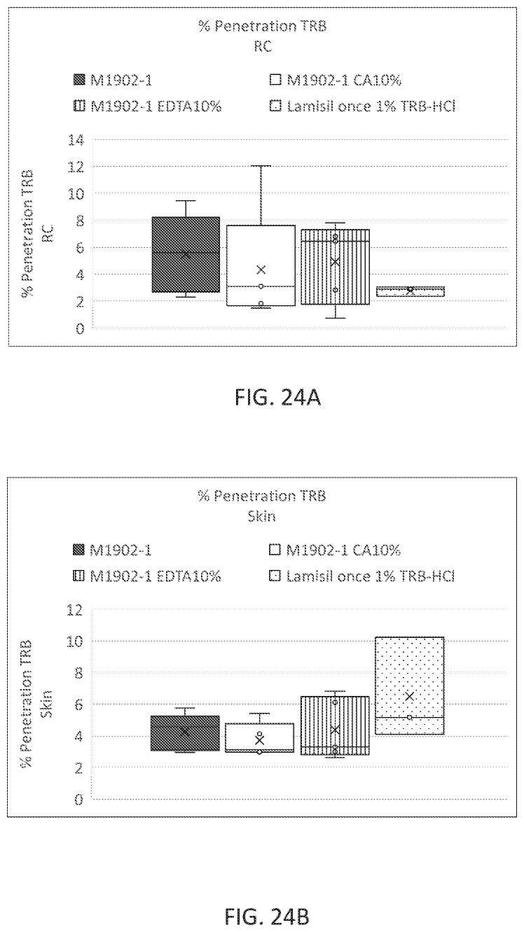

The influence of polymer crosslinking on permeation was also tested. FIG. 24A depicts TRB permeation (% from applied dose) into receptor cells and FIG. 24B, which depicts TRB permeation (% from applied dose) into skin for BJ-h nanodomains system embedded within different polymeric matrices, namely: PVA, CMC, poloxamer 407 and TR1 as polymers, sorbitol and glycerol as plasticizers and lactic acid and urea as penetrating enhancers (TRB concentration was 2.7 wt %) (M1902-1—dark gray bars) without cross-linking; M1902-1 with EDTA-mediated cross-linking (bars with vertical stripes); M1902-1 with citric acid-mediated cross-linking (white bars) as compared to Lamisil Once (doted bars). As seen from FIG. 24A greater permeation of TRB-HCl was observed into the receptor cell as compared to permeation into skin (both compared to the control). This suggests that the release capability of these films is greater in the receptor cell compared to the skin and thus that cross-linked films are advantageous for APIs in which primarily transdermal but also topical delivery is desired. Furthermore, it is noted that different cross-linking agents provide different degrees of crosslinking. It is further contemplated that by controlling the crosslinking degree, the films can be customized to provide a desired release profile (e.g. transdermal vs. topical and slow vs. fast release)

In general, it is noted that permeation of the API (here TRB-HCl) into the skin and receptor cells may be controlled by choosing specific film formers, nanodomain composition, by adding a permeating agent and/or by crosslinking of the films. Some films provided greater permeation into the skin (for example M43, M252, M123, M1319B and M211 (both nanodomains systems)). Other films were provided pronounced permeation into the receptor cell (for instance systems M1902-1 both crosslinked and not crosslinked).

Accordingly, when transdermal applications are required, films providing efficient permeability into the receptor cell are favorable whereas, when the target is the skin, films that provide high permeation into the skin are favored. That is, the adequate film is chosen based on the target.

### Example 5: In-Vivo Pharmacokinetic (PK) Study

In order to assess the levels of the API in the blood stream as well as its elimination and bioavailability, a PK is performed. In PK studies the API (within the delivery system of interest as compared to a control) is administrated to a group of participants (can be both animals or humans), and blood samples are taken, commonly, during 24 hours from the administration. The levels of the API are determined using high precision procedures (such as LC-MS/MS) and PK profile is plotted (API concentration vs time). Using mathematical models, several parameters are extracted from the PK curves including: Tₘₐₓ, Cₘₐₓ, AUC₀₋₂₄, AUC_{0-∞} and bioavailability or relative bioavailability.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1.-49. (canceled)

50. A drug delivery system comprising a soft polymeric film and a plurality of nanodomains embedded within the film, wherein the film, having embedded therein the nanodomains, wherein the nanodomains comprise less than about 25 wt % solvent and wherein the nanodomains maintain their structural integrity within the film when stored at room temperature for 1 month or more.

51. The drug delivery system of claim 50, wherein the nanodomains comprise less than about 35 wt % polyols.

52. The drug delivery system of claim 50, wherein the nanodomains comprise 30 wt %-60 wt % surfactant.

53. The drug delivery system of claim 50, wherein the nanodomains have an average size of less than about of 100 nm.

54. The drug delivery system of claim 50, wherein the nanodomains are loaded with an active pharmaceutical ingredient (API), wherein the film is configured to controllably release the API and/or the nanodomains upon being adhered, and wherein the release of the API from the film during storage is residual.

55. The drug delivery system of claim 50, wherein the film has a Young's Modulus elasticity in a range of about 0.1 KPa-1.5 MPa, and a tensile strength at breaking point in a range of about 0.4 KPa-1 MPa.

56. The drug delivery system of claim 50, wherein the nanodomains comprise 5-60 wt % of the total dry weight of the drug delivery system and the film polymer comprises 10-60 wt % of the total dry weight of the drug delivery system.

57. The drug delivery system of claim 50, wherein the film further comprises a plasticizer.

58. The drug delivery system of claim 50, wherein the polymeric film is configured to self-adhere to the subject's tissue surface.

59. The drug delivery system of claim 50, wherein the nanodomains comprise at least one hydrophilic surfactant, the at least one hydrophilic surfactant has a Critical Packing Parameter (CPP) of ⅓.

60. The drug delivery system of claim 59, wherein the at least one hydrophilic surfactant is selected from: polyoxyethylene, polyoxyethylene sorbitan monolaurate, polyoxyethylene sorbitan monopalmitate, polyoxyethylene sorbitan monostearate, polyoxyethylene sorbitan monooleate, a polyoxyethylene ester of saturated or unsaturated castor oil, an ethoxylated monoglycerol ester, an ethoxylated fatty acid, ethoxylated (20EO) sorbitan monolaurate (T20), ethoxylated (20EO) sorbitan monostearate/palmitate (T60/T40), ethoxylated (20EO) sorbitan mono oleate/linoleate (T80), ethoxylated (20EO), castor oil ethoxylated (20EO to 60EO); hydrogenated castor oil ethoxylated (20 to 60EO), ethoxylated (5-40 EO) monoglyceride stearate/palmitate, polyoxyl 35 castor oil, polysorbate 20 (Tween 20), polysorbate 40 (Tween 40), polysorbate 60 (Tween 60), polysorbate 80 (Tween 80), Mirj S40, Mirj S20, oleoyl macrogolglycerides, polyglyceryl-3 dioleate, ethoxylated hydroxyl stearic acid (Solutol HS 15), a sugar ester, a polyglycerol ester, ethoxylated castor oil, a polyglycerol ester, a mono- or di-glycerol ethoxylated fatty acid (20 to 40 EU), polyoxyethylene alkyl ethers (Brijs), and any combination thereof.

61. The drug delivery system of claim 50, wherein the nanodomains further comprise at least one lipophilic surfactant, the at least one lipophilic surfactant has a CPP of about 1.0-1.3.

62. The drug delivery system of claim 61, wherein the at least one lipophilic surfactant is selected from sorbitan, monoglyceride stearate, sorbitan monooleate, sorbitan tri stearate or tri oleate, a phospholipid, mono- or di-glycerides of fatty acids, a polyglycerol ester of stearic acid or palmitic acid or lauric or oleic and any combination thereof.

63. The drug delivery system of claim 50, wherein the nanodomains further comprise at least one of a short to medium chain alcohol, a co-solvent, a permeation agent, a membrane recognition agent, an antioxidant, a preservative, and any mixture thereof.

64. The drug delivery system of claim 50, wherein the nanodomains further comprise at least one solvent.

65. The drug delivery system of claim 64, wherein the at least one solvent is selected from: medium-chain triglyceride (MCT), olive oil, soybean oil, peanuts oil, canola oil, cotton oil, palmolein, sunflower oil, corn oil, pumpkin oil, moringa oil, cannabis oil, sesame oil, grape seeds oil, avocado oil, pomegranate seeds oil, neem oil, lavender oil, peppermint oil, anise oil, ginger oil, isopropyl myristate (IPM), isopropyl palmitate (IPP), oleyl lactate, coco caprylate, hexyl laurate, oleyl amine, oleic acid, oleyl alcohol, linoleic acid, linoleyl alcohol, ethyl oleate, hexane, heptane, nonane, decane, dodecane, D-limonene, neem oil, lavender oil, peppermint oil, anise oil, menthol, capsaicin, dimethicone, cyclomethicone or any combination thereof.

66. The drug delivery system of claim 50, wherein the nanodomains are distributed evenly over and within the film.

67. The drug delivery system of claim 50, wherein the film is made of gelatin, gelatin and polyvinylpyrrolidone, gelatin and poloxamers, polyvinyl alcohol, hydroxylethyl cellulose, hydroxypropyl cellulose, methyl cellulose, carbomers, chitosan, pectin, hyaluronic acid, or any combination thereof.

68. The drug delivery system of claim 54, wherein the nanodomains comprise 2-20% w/w of API.

69. The drug delivery system of claim 54, wherein at least 0.01% of the API is released from the film 1 day after being adhered to the subject's skin.

70. The drug delivery system of claim 54, wherein the API is a pharmaceutical, a cosmetic, a cosmeceutical, a nutraceutical, or any combination thereof.
